(19) United States
(12) Patent Application Publication
(10) Pub. No.: US 2026/0256876 A1
(43) Pub. Date: Sep. 3, 2026

(54) ANTIMICROBIAL RECOMBINANT LIVE PRODUCTS AND METHODS

(71) Applicant: GENERAL PROBIOTICS, INC., St. Paul, MN (US)

(72) Inventors: Yiannis N. Kaznessis, St. Paul, MN (US); Kathryn Gayle Kruziki, St. Paul, MN (US); Samuel Weber Morris, St. Paul, MN (US)

(21) Appl. No.: 18/878,459

(22) PCT Filed: Jun. 28, 2023
(86) PCT No.: PCT/US2023/069266

§ 371 Date: Dec. 23, 2024

Related U.S. Application Data
(60) Provisional application No. 63/367,559, filed on Jul. 1, 2022.

(51) Int. Cl. A61K 38/16; A61K 35/742; A61P 1/00; A61P 31/04; C12N 1/205; C12N 15/75; C12R 1/10

(52) U.S. Cl. CPC A61K 38/164; A61K 35/742; A61P 1/00; A61P 31/04; C12N 1/205; C12N 15/75; C12R 2001/10

(57) ABSTRACT

This disclosure relates to antimicrobial recombinant live therapeutics that are Bacillus species plural (spp.) that colonize the gastrointestinal tract of animals. The Bacillus live therapeutics are genetically modified to produce antimicrobial peptides such as class II bacteriocins. The Bacillus live therapeutics form spores and are fed to animals. The Bacillus live therapeutics lower carriage of Clostridia perfringens (C. perfringens) in fed animals and are used to prevent and control disease conditions caused by C. perfringens, for example, necrotic enteritis.

### CROSS-REFERENCE TO RELATED APPLICATIONS

This application claims priority to U.S. Provisional Application No. 63/367,559, filed Jul. 1, 2022, which is incorporated by reference herein in its entirety.

### Incorporation by Reference of the Sequence Listing

This application contains, as a separate part of disclosure, a Sequence Listing in computer-readable form (Filename: 57699A_SeqListing.xml; 95,557 bytes-XML file dated Jun. 13, 2023) which is incorporated by reference herein in its entirety.

### FIELD

This disclosure relates to antimicrobial recombinant live therapeutics that are Bacillus species plural (spp.) that colonize the gastrointestinal tract of animals. The Bacillus live therapeutics are genetically modified to produce antimicrobial peptides such as class II bacteriocins. The Bacillus live therapeutics form spores and are fed to animals. The Bacillus live therapeutics lower carriage of Clostridia perfringens (C. perfringens) in fed animals and are used to prevent and control disease conditions caused by C. perfringens, for example, necrotic enteritis.

### BACKGROUND

There are an estimated billion people who are malnourished because of the absence of animal protein in their diets. There are an estimated 130 million children whose growth has been stunted because they lack access to milk, eggs and meat.

Despite efforts to grow alternative protein without animals, animal protein is and will remain a critical component of diets globally. An increasing human population and improving standards of living are resulting in higher demand for animal protein globally.

While demand increases, supply of animal protein faces mounting challenges. A confluence of environmental factors, including climate instabilities and water scarcity, and risks of life-threatening, zoonotic pandemics, are exerting pressure on global food security.

An additional, significant pressure results from the removal of antibiotics from livestock production. Farmers have long depended on antibiotics to raise healthy animals, free of foodborne pathogens. But the overuse of antibiotics in agriculture may be resulting in the selection and emergence of bacterial pathogens with resistance to all classes of available antibiotics.

The risk of resistance to antibiotics is resulting in a global push to remove antibiotics from agriculture. This may be a good step in the right direction in terms of mankind's ability to fight bacterial infections. But without antibiotics animals get sick with bacterial infections. Sick animals result in lower supply of animal protein. Sick animals result in wasted resources and negative environmental impact. Sick animals result in contaminated food that sickens humans.

Focusing on the poultry industry, phasing out of antibiotics is resulting in higher frequency of necrotic enteritis (NE). The causative agent of NE is Clostridia perfringens, a Gram-positive anaerobe. NE causes a significant negative economic impact in broiler production. The acute form of NE leads to increased mortality in the broiler flocks, reaching 1% losses per day for consecutive days. Estimates of $6 billion annually have been reported as the economic worldwide impact of NE on the global poultry industry.

It is estimated that without antibiotics, 700 million additional birds will need to be raised to meet poultry demand in the United States annually, requiring approximately 2 billion additional gallons of water and 5.4 million additional tons of feed per year.

C. perfringens in poultry is also a major risk for foodborne transmission to humans. Type A and type C. C. perfringens strains cause type A diarrhea and type C necrotic enteritis, respectively, in humans. Approximately 1 million cases of C. perfringens infections are reported annually in the United States

As defined by The Food and Agriculture Organization of the United Nations (FAO), “probiotics” are “live microorganisms that, when administered in adequate amounts, confer a health benefit on the host.” Probiotic bacteria and yeast are among the leading alternatives to antibiotics in livestock and are believed to elicit numerous benefits to animal health and production. These supposed benefits include growth promotion, improvement in gut integrity, and the reduction of intestinal infections, such as necrotic enteritis. Consequently, animal producers have turned to probiotics to deter intestinal pathogens.

One of the primary proposed mechanisms of action of probiotics against intestinal pathogens is their production of antimicrobial peptides. Antimicrobial peptides are small proteins, typically between about 10 and about 100 amino acids in length that inhibit, and often kill, certain microbes. Bacteriocins are antimicrobial peptides that are naturally produced by bacteria.

Bacteriocin production is an extremely common trait among bacteria and likely evolved in part to enable the producer strain to better compete against invading microbes for resources.

The delivery of antimicrobial peptides using engineered probiotics has been previously proposed for the treatment of various pathogens including Salmonella, Vancomycin-resistant enterococci, and Pseudomonas aeruginosa [Forkus et al., Scientific Reports 7, 40695 (2017); Geldart et al., Bioengineering & Translational Medicine 3, 197-208 (2018); Hwang et al., Nature Communications 8, 15028 (2017)].

For example, Forkus et al., supra shows that E. coli Nissle 1917 can be used to produce microcin J25 using the native microcin J25 secretion system. This secretion system however is unable to be used in Gram-positive bacteria.

Borrero et al. [ACS Synthetic Biology, 4, 299-306 (2015)] and Geldart et al. [Applied and Environmental Microbiology, 81, 3889-3897 (2015)] show that enterocin A can be secreted from Lactococcus lactis using the L. lactis-derived Usp45 secretion tag. The Usp45 tag could not be used to secrete a different peptide, microcin V, from L. lactis.

Kaznessis et al. (WO 2021/154872 A1) show that enterocin A can be produced from Escherichia coli inside the gastrointestinal tract of animals.

Bacillus species are widely used as probiotic organisms in both the food and animal health industries. Several Bacillus species are considered to be “Generally Recognized as Safe” (GRAS) by the Food and Drug Administration. Species or strains that have attained GRAS status are considered safe for use in food by a panel of experts and are thus preferred for use in livestock compared to non-GRAS organisms. Bacillus species that have attained GRAS status include; Bacillus subtilis, Bacillus coagulans, Bacillus licheniformis, and Bacillus pumilus. Additional species that are commonly used as probiotics include Bacillus clausii and Bacillus amyloliquefaciens [Susanti et al., Frontiers in Microbiology, 12, 3116 (2021)].

Bacillus species are known to produce a plethora of bacteriocins [Abriouel et al., FEMS Microbiology Reviews, 35, 201-232 (2011)]. Furthermore, several strains have been reported to produce bacteriocins with in vitro activity against C. perfringens [Teo and Tan, Applied and Environmental Microbiology, 71, 4185-4190 (2005); Hyun et al., LWT, 138, 110625 (2021); Caly et al., Frontiers in Microbiology, 6, 1336 (2015); Ghanbari et al., Iranian Journal of Veterinary Research, 10, 267-272 (2009)].

Clostat (Bacillus subtilis PB6) and DSM 29870 are two of the few Bacillus-based products with both reported in vitro anti-Clostridial activity and in vivo efficacy against NE-induced mortality in poultry [Jayaraman et al., Poultry Science, 96, 2614-2622 (2017); Teo and Tan, Journal of Applied Poultry Research, 15, 229-235 (2006); Teo and Tan, Journal of Applied Poultry Research, 16, 296-303 (2007); WO2016/118840]. Clostat, however, exhibits hemolytic activity which may indicate toxigenic behavior [US-2019-0091269].

Bacillus species, and Bacillus subtilis in particular, are commonly engineered for the production and secretion of heterologous proteins [Su et al., Microbial Cell Factories, 19, 1-12 (2020); Cai et al., Journal of Applied Microbiology, 126, 1632-1642 (2019)]. One study has shown the heterologous production of a bacteriocin out of a Bacillus species. In this lone study, the authors simply transferred the native bacteriocin gene cluster for thurincin H from one strain of Bacillus thuringiensis to another strain of Bacillus thuringiensis [Wang et al., Journal of Dairy Science, 97, 4115-4119 (2014)].This study thus involves the intraspecies transfer of a gene cluster and includes no modification of the naturally-occurring gene cluster.

Thus, there remains a need in the art for antimicrobial recombinant live therapeutics and methods for their use.

### BRIEF SUMMARY

Engineering Bacillus isolates to reliably produce anti-Clostridial bacteriocins is contemplated herein to provide effective, safe antimicrobial recombinant live therapeutics (i.e., antimicrobial recombinant live bacterium). Alternatively, these antimicrobial recombinant live bacterium are contemplated for use in the industrial production of antimicrobial peptides.

The disclosure herein relates to the isolation (for example, from the small intestinal tract of healthy chickens), characterization, screening, selection, and engineering of Bacillus spp, for the treatment of disease conditions caused by C. perfringens such as necrotic enteritis, for example, in poultry. For example, a composition for treatment of broiler chickens is provided. The composition can comprise Bacillus spp. spores. The composition can be incorporated in the feed of chickens or in the water.

The antimicrobial recombinant live therapeutics provided herein are genetically engineered with an exogenous polynucleotide that can include a secretion tag sequence, a heterologous promoter and a polynucleotide that encodes an antimicrobial protein such as a class II bacteriocin. The antimicrobial peptide is contemplated to be effective in killing C. perfringens inside the gastrointestinal tract of animals.

Thus, the disclosure provides a composition for treating a Clostridia perfringens-induced disease condition. The composition comprises a Bacillus bacterium isolated from an intestinal tract of an animal and transformed with an exogenous polynucleotide, wherein the exogenous polynucleotide comprises a heterologous promoter operably linked to a polynucleotide that encodes a secretion tag fused to an antimicrobial protein with bacteriolytic or bacteriostatic activity against Clostridia perfringens. An exemplary Clostridia perfringens-induced disease contemplated is necrotic enteritis.

The Bacillus bacterium provided can be a Bacillus spp. strain bacterium, for example, a Bacillus oleronius strain GP01252 bacterium transformed with the exogenous polynucleotide; a Bacillus licheniformis, Bacillus paralicheniformis. Bacillus oagulans, Bacillus pumilus, Bacillus clausii, or Bacillus anyliquefaciens transformed with the exogenous polynucleotide; or a Bacillus paralicheniformis strain GP01336 bacterium (ATCC Accession No. PTA-127307) transformed with the exogenous polynucleotide.

The Bacillus bacterium provided may lack genes of proteases NprE, NprB, AprE, WprA, Vpr, Bpr, Epr, HtrA, and/or HtrB with greater than 70%, greater than 60%, or greater than 50% homology to the corresponding genes present in Bacillus subtilis 168.

The Bacillus bacterium provided may contain genes for the expression of Class I bacteriocins.

The antimicrobial peptide expressed from the exogenous polynucleotide with which the Bacillus bacterium is transformed can be a class II bacteriocin such as enterocin A, enterocin B, enterocin P, carnobactreiocin B, plantaricin EF, or hiracin JM79.

The heterologous promoter driving expression of the antimicrobial peptide from the exogenous polynucleotide can be the constitutive promoter p43. The exogenous polynucleotide can be a plasmid.

The recombinant Bacillus bacterium provided can be a thymidine auxotroph comprising the thymidylate synthase A (ThyA) gene under the control of a heterologous promoter such as the p43, pylB, or pgsiB promoter, which can exert selective pressure on the exogenous plasmid.

The recombinant Bacillus bacterium provided can be a prototroph and the selective pressure on the exogenous plasmid can be exerted by a kanamycin resistance gene.

Antimicrobial recombinant live therapeutic compositions provided herein can further comprise a pharmaceutically acceptable carrier.

Methods are provided for treating a disease condition caused by Clostridia perfringens in an animal, comprising administering to the animal in need thereof, an antimicrobial recombinant live therapeutic composition of the disclosure. The animal can be: a mammal such as a human, dog, cat or pig; a bird such as a chicken, turkey or duck; or a fish.

Methods are provided for restoring rate of weight gain in an animal that has necrotic enteritis caused by Clostridia perfringens, comprising the step of administering to an animal in need thereof, an antimicrobial recombinant live therapeutic composition of the disclosure. The animal can be: a mammal such as a pig; or a bird such as a chicken, turkey or duck; or a fish.

### BRIEF DESCRIPTION OF THE DRAWINGS

FIG. 1 depicts a protease assay assessing the proteolytic/inactivation activity of ten Bacillus intestinal isolates against enterocin A along with B. subtilis 168 and E. coli. The activity of these isolates was compared to that of GP0700, an E. coli SI isolate, which is known to have minimal proteolytic activity towards enterocin A. Bacillus spp. isolates 3, 4, and GP01252 as well as B. subtilis 168 exhibit high proteolytic activity. In contrast, Bacillus spp. isolate 2 and GP01336 exhibited the lowest proteolytic activity against enterocin A.

FIG. 2 is a schematic of an exemplary plasmid used to engineer GP01415 for the expression of enterocin A. The plasmid contains the transcriptional units for thyA and enterocin A. In this case, thyA expression is driven by the p43 and enterocin A uses the AmyQ secretion tag. The repB gene encodes the Bacillus replication protein that allows for a high plasmid copy number in GP01415. The plasmid also contains an ampicillin resistance gene and origin of replication for E. coli to allow for plasmid construction in an E. coli MC1061 F′. The sequence of the plasmid is set out in SEQ ID NO: 44.

FIG. 3 is a schematic of representative antimicrobial peptide transcriptional units. In the first example, the promoter in front of the enterocin A sequence is the p43 (constitutive) promoter, the ribosome-binding site (RBS) is R0 and the terminator is B0015. Theoretically, different combinations of the promoters, RBSs, secretion tags, and mature antimicrobial peptide sequences can be used to produce the most efficient construct for a given target organism as seen in the other two constructs.

FIG. 4 is a group of agar diffusion assays of some of the engineered systems expressing and secreting enterocin A. A) Depicts a group of systems using GP01415 (rifampicin-resistant thymidine auxotroph of GP01336) as a chassis. B) Depicts engineered systems GP01270 and GP01284 derived from GP01252. The secretion tags as well as the type of selective pressure for each system can be found in Table 5. The indicator strain E. facieum 8E9 is seeded in BHI at a concentration of 0.5 μL/mL.

FIG. 5 is an example of a stab-on-agar assay testing the effectiveness GP01252 (left) and GP01336 (right) engineered with enterocin A using library of secretion tags that are fused to a mature enterocin A sequence. The variability in the sizes of zones of inhibition demonstrates the importance of secretion tag on peptide production. Comparatively, the halos of engineered isolates from GP01336 are larger than those derived from GP01252 and the GP01336 isolates with the largest halos (indicated by the white arrows) were sequenced to identify the secretion tag.

FIG. 6 is a minimum inhibitory concentration (MIC) assay using the supernatant (SN) of GP01416 against E. faecium 8E9. GP01336 shows no SN activity towards 8E9. The negative control contains no supernatant. GP01191 is an enterocin A-producing E. coli strain that has been successful in reducing C. perfringens-related necrotic enteritis in chickens. The purified enterocin A was used to quantify the amount of enterocin A produced by GP01416.

FIG. 7 is an MIC assay with the same setup as FIG. 6 but using L. monocytogenes as the indicator strain.

FIG. 8 is an agar diffusion assay demonstrating antimicrobial activity against various C. perfringens strains. A) Depicts stab-on-agar assay of recombinant live therapeutic GP01416 against the following C. perfringens strains: CP #39, NAH-JP1011, CP #26, and SPRG #6. GP01336 (indicated by black dots on some of the plates) shows little to no inhibition against C. perfringens depending on the strain. GP01416 consistently displays larger halos compared to GP01336. B) Depicts stab-on-agar assay of GP01252 with its modified counterpart GP01270 against NAH-JP1011.

FIG. 9 depicts the growth of GP01336 compared to GP01416 in rich media (Brain Heart Infusion Broth) and in Gl-tract contents (jejunal contents) versus time. Tween 80 at a concentration of 0.075% was added to media to reduce aggregation. Growth was monitored using optical density measured at 600 nm.

FIG. 10 shows an example of a stab-on-agar assay testing the effectiveness of unmodified Bacillus strains and of recombinant strains that have been modified with plasmids that express and secrete enterocin A (EntA). The larger the halo size the more enterocin A is produced and secreted by each strain. GP01252 and GP01336 are strong EntA producers because they lack the proteases present in other Bacillus strains.

Bacterial strain names in the Figures which do not include a zero as the third character refer to the same bacterial strains as the foregoing figure descriptions which include names with a zero as the third character (e.g., GP1252 refers to the same bacterial strain as GP01252).

### DETAILED DESCRIPTION

Using a novel approach to strain selection, Bacillus bacterium provided herein are selected and genetically engineered to produce and secrete heterologous antimicrobial peptides. These genetically engineered strains can then be used to reduce pathogens in the intestinal tract of animals.

The present disclosure is based, at least in part, on findings showing that not all bacteria isolated from the gastrointestinal tract of animals can be engineered to re-colonize the gut of animals and maintain the metabolic activity for protein production. Bacillus isolates provided herein are selected based on localization and metabolic activity in intestinal tract conditions.

The present disclosure is also based, at least in part, on findings showing that not all bacteria isolated from the gastrointestinal tract of animals can be engineered to express and secrete antimicrobial peptides because of specific intracellular and extracellular proteases encoded in the genome of the bacteria that enzymatically digest antimicrobial peptides. Bacillus isolates provided herein are selected based on the absence of such proteases from their genome.

Bacillus isolates provided herein can be further selected based on the absence of virulence factors, other pathogenicity factors, and/or antibiotic resistance.

The Bacillus isolates selected herein are then genetically engineered to express antimicrobial peptides.

Numerous tools and protocols exist for genetic engineering of Bacillus, many of which were originally developed for the model strain, Bacillus subtilis 168 [Guiziou et al., Nucleic Acids Research, 44, 7495 (2016); BGSC-Home, bgsc.org].

Exogenous polynucleotides are engineered to contain a bacterial promoter, a ribosome binding site, a secretion tag encoding region, a antimicrobial peptide encoding region, and a terminator sequence. The antimicrobial peptide encoded can be a class II bacteriocin.

Bacillus isolates are transformed with libraries of the exogenous polynucleotide constructs. The recombinant Bacillus isolates are screened for antimicrobial activity in vitro to evaluate peptide production. The recombinant Bacillus isolates are tested for in vitro activity against the pathogen of interest, for example, C. perfringens

The genetically engineered recombinant Bacillus isolates are tested for in vivo activity against the pathogen of interest. The host animal can be of the class Aves. The host animal can be of the species Gallus gallus domesticus (chicken). The pathogen of interest can be C. perfringens The diseases to be alleviated include, but are not limited to, necrotic enteritis.

Isolation of Bacillus spp. from Chicken Intestines

Bacilli can be isolated from the intestines of healthy birds as described, e.g., in Example 1. Bacillus spp. can be isolated from the jejunal, ileal, or cecal regions of the intestinal tracts of healthy birds. Bacillus spp. can be isolated from, for example, the luminal contents of the intestinal tract or the epithelial mucus layer of the intestinal tract.

The Bacillus spp. can be isolated from birds challenged, for example, with Eimeria spp, or with Clostridium perfringens.

Bacillus spp. can be isolated from chicken breeds including, but not limited to, Cornish Cross, Orpington, Freedom Rangers, White leghorn, Delaware, Marans, Welsummer and Buckeye. Bacillus spp. can be isolated from the intestines of chickens bred and marketed by Aviagen corporation including, but not limited to, Ross, Rowan Range and Specialty Males brands. Bacillus spp. can be isolated from the intestines of chickens bred and marketed by Cobb-Vantress corporation including, but not limited to, Cobb500, Cobb70, MV Male and Vantage Male brands. Bacillus spp. can be isolated from the intestines of chickens bred and marketed by Hubbard corporation including, but not limited to, Hubbard efficiency and Hubbard efficiency plus brands.

Exemplary isolated Bacilli include the strains designated GP001252 and GP001336. Bacillus paralicheniformis strain GP01336 was deposited on May 24, 2022 with the American Type Culture Collection (ATCC), 10801 University Boulevard, Manassas, Virginia 20110, USA, and was assigned ATCC Accession No. PTA-127307. Viability of the GP01336 deposit was tested and confirmed by the ATCC on Jun. 30, 2022.

Characterization of Various Bacillus spp. from Chicken Intestines

Bacillus spp. colonies can be tested using DNA fingerprinting colony PCR (cPCR) to identify unique isolates, as described for example in Example 2.

Bacillus spp. can be characterized using 16S ribosomal RNA sequencing, as described, e.g., in Example 3.

Bacillus spp. isolated according to the disclosure include, for example, Bacillus subtilis, Bacillus licheniformis, Bacillus paralicheniformis, Bacillus coagulans, Bacillus pumilus, Bacillus clausii, or Bacillus anyliquefaciens.

Isolated Bacillus spp. can be tested for the presence of virulence and pathogenic factors, including but not limited to, T7SS, ALO, anthrax toxin, cerulide, certhrax, cytK, HBL, nhe, inhA, capsule, bacillibactin, hal, ilsA, petrobactin, atxA, and bslA. Bacillus spp. are selected based on the absence of these factors.

Isolated Bacillus spp. can be tested for antimicrobial peptide inactivation (referred to as protease activity), as described, e.g., in Example 4. Bacillus spp. are selected for low protease activity.

Isolated Bacillus spp. can be tested for the presence of intracellular, membrane and extracellular proteases, including but not limited to, nprE, nprB, AprE, WprA, Vpr, Bpr, Epr, HtrA, and HtrB. Bacillus spp. can be selected for the absence of these proteases.

Isolated Bacillus spp. can be tested for innate antimicrobial activity against Gram+strains, including but not limited to, Enterococcus faecium 8E9 and Listeria monocytogenes CDC 7762. Bacillus spp. can be selected for high antimicrobial activity against Gram+microbes, as described, e.g., in Example 5.

The isolated Bacillus spp. can be tested for susceptibility to the antimicrobial peptide of interest. Examples of antimicrobial peptides include, but are not limited to, carnobacteriocin A, enterocin A, enterocin B, and hiracin JM79. Bacillus spp. are selected for not being susceptible to bacteriocins, as described, e.g., in Example 6.

The isolated Bacillus spp. can be tested for their susceptibility to antibiotics, including but not limited to, kanamycin, chloramphenicol, tetracycline, enrofloxacin, and amoxicillin. Bacillus spp. can be selected for being susceptible to antibiotics as described, e.g., in Example 7.

The isolated Bacillus spp. can be tested for hemolytic activity. Bacillus spp. can be selected for not exhibiting hemolytic activity, as described, e.g., in Example 8.

Transformation of Bacillus Spp. Isolates with Exogenous Polynucleotides

Bacillus spp. can be transformed with a recombinant exogenous polynucleotide (including, but not limited to, a construct such as a recombinant DNA plasmid) as described, e.g., in Example 9. See Example 10 for a description of an exemplary plasmid design.

### Plasmid Backbone/Architecture

The plasmids used to transform Bacillus contain an origin of replication. The plasmid can contain an E. coli origin of replication. The plasmid can contain a Bacillus origin of replication such as the Bacillus origin of replication pUB110 or derivatives thereof.

The plasmids contain a selectable marker for the recombinant Bacillus spp. The selectable marker can be a kanamycin resistance gene. The selectable marker can be a functional dapA or thyA gene isolated from a Bacillus spp. isolate.

The plasmids contain a promoter region linked to one or more ribosome binding sites which is/are linked to the secretion tag/antimicrobial peptide gene fragment fusion region (the coding region) which is linked to the terminator. This polynucleotide thus comprises the antimicrobial peptide transcriptional unit (TU). The TU encompasses both the coding region and the regulatory sequence. The TU can polycistronic and includes the secretion tag/antimicrobial peptide along with an additional secretion tag/antimicrobial peptides or other genes of interest.

Exemplary final DNA plasmids are pKG293, pSM504, pSM547-pSM552, pSM576, pSM583, pSM584, pSM587, or pSM595-pSM600.

### Promoters

The promoter used to express the secretion tag/antimicrobial peptide fusion can be, for example, the Bacillus p43 constitutive promoter or variants thereof, the Bacillus pveg constitutive promoter or variants thereof, the Bacillus pgsiB promoter or variants thereof, the Bacillus pyIB promoter or variants thereof, the Bacillus pxylA promoter or variants thereof, or the Bacillus plial promoter or variants thereof.

Naturally occurring bacteria monitor environmental conditions and they respond by modifying the expression pattern of their genes. Transcription of genes is carried out by a single species of RNA polymerase (RNAP). The core enzyme of RNAP executes RNA polymerization reactions, but it cannot recognize a DNA promoter, bind to it and initiate transcription.

The task of promoter recognition in bacteria is left to one of a few protein subunits called sigma factors. Each sigma factor binds to its cognate promoter and connects with the RNAP core enzyme, forming the fully functioning RNAP holoenzyme. In E. coli there are seven known sigma factors and each bind to DNA promoters under different conditions. For example, Sigma 70 binds to its cognate DNA promoters at all times. Sigma 38 binds to its DNA cognate promoters in stationary state.

Thus, expression of a gene of interest can be controlled by employing promoters that interact with sigma factors that are dominant under the desired expression condition. For example, by employing a promoter capable of binding sigma 38 but not sigma 70, gene expression would be upregulated in stationary phase rather than in exponential phase.

A list of known sigma factors active in vegetative Bacillus spp. is presented in Table 1.

TABLE 1
Known sigma factors in Bacillus spp.

| Sigma Factor | Gene | Purpose of Regulation |
|---|---|---|
| σ^{A} | sigA, rpoD | Housekeeping |
| σ^{B} | sigB | General stress |
| σ^{H} | sigH, spoOH | Postexponential, |
| | | competence, and early |
| | | sporulation |
| σ^{D} | sigD, flaB | Chemotaxis, autolysin, and |
| | | flagellar control |
| σ^{L} | sigL | Degradative enzyme control |

Table 2 provides a list of exemplary promoters compatible with various Bacillus spp.

TABLE 2
Examples of Bacillus spp. promoters

| | | | |
|---|---|---|---|
| Promoter | Description | Sequence | Article |

Constitutive

| | | | |
|---|---|---|---|
| pveg | Strong constitutive | SEQ ID NO: 23 | Radeck et al., |
| | promoter | | Journal of |
| | | | Biological |
| | | | Engineering, 7, |
| | | | (2013) |
| p43 | Constitutive promoter | SEQ ID NO: 24 | Wang and Doi, |
| | | | The Journal of |
| | | | Biological |
| | | | Chemistry, 259, |
| | | | 8619-8625 (1984) |

Stationary Phase-Induced

| | | | |
|---|---|---|---|
| pgsiB | Induced by heat, | SEQ ID NO: 25 | Paccez et al., |
| | ethanol, acid shock as | | Vaccine, 24, 2935- |
| | well as glucose and | | 2943 (2006); |
| | phosphate starvation | | Nguyen et al., |
| | | | Plasmid, 54, 241- |
| | | | 248 (2005) |
| pylB | Induced by Rhamnose, | SEQ ID NO: 26 | Yu et al., Scientific |
| | Repressed by Glucose | | Reports 5: 18405, |
| | | | 1-9 (2015) |

Inducible

| | | | |
|---|---|---|---|
| pxyla | Xylose-inducible | SEQ ID NO: 27 | Popp et al., |
| | | | Scientific Reports, |
| | | | 7: 15058, 1-13 |
| | | | (2017) |
| plial | Bacitracin-inducible | SEQ ID NO: 28 | Popp et al., supra |

The pveg promoter in B. subtilis is a super constitutive promoter and is the strongest known promoter in Bacillus. It is an σ^{A}-dependent promoter that seems to be essential for the growth of germinating cells coming out of the sporulation stage. Pveg is a popular promoter to use for heterologous protein production, both plasmid and genome-based, due to its strong activity.

The p43 promoter is another popular constitutive promoter used for heterologous protein production in Bacillus. One reason why p43 is a popular promoter to use is because its region has overlapping binding sites for σ^{A} and σ^{B} which make it active during both exponential and stationary phase. Studies of heterologous protein production with p43 have proven that the target protein is produced from the start of exponential phase all the way to late stationary phase. P43, due to its constitutive nature, does not appear to be an attractive promoter to express antimicrobial proteins due to toxicity risks to the producer cells if the cell is also susceptible to the protein.

Two popular stationary phase-induced promoters to use for heterologous protein production include the pgsiB and pylB. The promoters are desirable for industry use because they are auto-inducible promoters which means an inducer does not need to be added during manufacturing resulting in reduced production costs. PgsiB is regulated by OB and is induced by heat shock, salt or ethanol stress, and glucose limitation. A study investigating the activity of pgsiB promoter under different environmental conditions showed that induction increased 7, 8, and 12-fold under acid, heat, and ethanol shock, respectively. It can be activated as early as mid-exponential phase and reach its peak activity at stationary phase. Additionally, pgsiB has low basal activity when it is not induced and could make it an attractive promoter to use to control the expression of a given target protein.

PylB is another common auto-inducible promoter that is being explored for industry uses. The promoter is most active during mid-exponential phase all the way through stationary phase. The expression level of pylB is directly correlated with the cell density. Overall, pylB has been shown to produce more of a target protein during exponential and stationary phase than the constitutive promoter p43.

Common inducible promoters in B. subtilis that are used in engineering genetic constructs include pxyla and plial. Pxyla is a promoter that is induced by xylose. The promoter is accompanied with a gene called xyIR which acts as the repressor on the promoter. In the presence of xylose, the repressor is released and the promoter is activated through derepression. The lial promoter is regulated by the LiaRS two-component system that responds to presence of bacitracin in sub-lethal amounts. It has a very low level of basal activity and is activated in a concentration-dependent manner.

### Ribosome Binding Sites

The ribosome binding site in a plasmid can be, for example, R0 and variants thereof, R1 and variants thereof, R2 and variants thereof, R3 and variants thereof, R4 and variants thereof, R5 and variants thereof, R6 and variants thereof, and/or R7 and variants thereof.

Gene expression can be controlled both through manipulation of the transcription efficiency as well as manipulation of the translation efficiency. The ribosome binding site (RBS) largely dictates the efficiency with which the transcribed mRNA is translated into a protein. Numerous ribosome binding sites have been identified for Bacillus subtilis 168. Sequences and libraries of these RBSs are publicly available.

Guiziou et al., supra developed multiple libraries of ribosomal binding sites and characterized their relative gene expression in Bacillus subtilis 168. Table 3 provides a list of exemplary RBSs and their sequences as described by Guiziou et al, supra. These RBSs and derivatives thereof can be used in various Bacillus spp. provided herein to control translation of genes of interest.

TABLE 3
Exemplary RBSs used in Bacillus spp.

| RBS Name | DNA Sequence (5′-3′) | SEQ ID NO |
|---|---|---|
| R0 | GATTAACTAATAAGGAGGACAAAC | 46 |
| R1 | GCTCTTAAGGAGGATTTTAGA | 47 |
| R2 | GGTGGAAAGGAGGTGATCGAC | 48 |
| R3 | GGTGGGAAGGAGGACATTCGAC | 49 |
| R4 | TGACATGAAAGGAAGTATTTGAAA | 50 |
| R5 | GGTGGGAAGGAGGTGATCCA | 51 |
| R6 | GGTGGGAAGGAGGGGGTTCGAC | 52 |
| R7 | GGTGGGAAGGAGGAACTACT | 53 |

### Antimicrobial Peptides

Antimicrobial peptides encoded by exogenous polynucleotides/constructs herein are peptides such as a bacteriocin. The bacteriocin can be derived from a Gram-positive bacterial species. The bacteriocin can be derived from a Gram-negative bacterial species (also referred to as a microcin). The bacteriocin can be a class II bacteriocin derived from a Gram-positive bacterial species including, but not limited to, a bacteriocin derived from Enterococcus spp (also referred to as an enterocin) such as enterocin A, enterocin P, hiracin JM79, enterocin B, and/or enterocin 96 or variants thereof; a bacteriocin derived from Carnobacterium spp (also referred to as a carnobacteriocin) such as carnobacteriocin A, divergicin A, and/or carnobacteriocin B2 or variants thereof; a bacteriocin derived from Lactococcus spp, such as garvicin A or variants thereof; or a bacteriocin derived from LactoBacillus spp, such as plantaricin E, plantaricin F, plantaricin J, and/or plantaricin K or variants thereof.

An antimicrobial peptide can be naturally occurring or can be engineered. Antimicrobial peptides are produced by all classes of organisms, including mammals, bacteria, and phage. Examples of antimicrobial peptides and their amino acid sequences are shown in Table 4.

TABLE 4
Exemplary antimicrobial peptides

| Antimicrobial | | |
|---|---|---|
| Peptide | Amino Acid Sequence | Origin |
| Enterocin A | TTHSGKYYGNGVYCTKNKCTVDW | E. faecium |
| (EntA) | AKATTCIAGMSIGGFLGGAIPGKC | [Aymerich et al., Applied and |
| | (SEQ ID NO: 1) | Environmental Microbiology, 62, |
| | | 1676-1682 (1996)] |
| Enterocin P | ATRSYGNGVYCNNSKCWVNWGE | E. faecium |
| (EntP) | AKENIAGIVISGWASGLAGMGH | [Cintas et al., Applied and |
| | (SEQ ID NO: 2) | Environmental Microbiology, 63, |
| | | 4321-4330 (1997)] |
| Enterocin B | ENDHRMPNELNRPNNLSKGGAKC | E. faecium |
| | GAAIAGGLFGIPKGPLAWAAGLAN | [Casaus et al., Microbiology |
| | VYSKCN (SEQ ID NO: 3) | (Reading, England) 143 ( Pt 7), |
| | | 2287-2294 (1997)] |
| Hiracin JM79 | ATYYGNGLYCNKEKCWVDWNQA | E. hirae |
| (HirJM79) | KGEIGKIIVNGWVNHGPWAPRR | [Sanchez et al., FEMS |
| | (SEQ ID NO: 4) | Microbiology Letters, 270, 227- |
| | | 236 (2007)] |
| Carnobacteriocin | DQMSDGVNYGKGSSLSKGGAKC | Carnobacterium piscicola LV17A |
| A | GLGIVGGLATIPSGPLGWLAGAAG | [Worobo et al., Microbiology |
| | VINSCMK (SEQ ID NO: 5) | (Reading, England), 140 ( Pt 3), |
| | | 517-526 (1994)] |
| Carnobacteriocin | VNYGNGVSCSKTKCSVNWGQAF | Carnobacterium piscicola LV17A |
| B2 | QERYTAGI NSFVSGVASGAGSIG | [Quadri et al., Journal of |
| | RRP (SEQ ID NO: 6) | Biological Chemistry, 269, |
| | | 12204-12211 (1994)] |
| Divergicin A | AAPKITQKQKNCVNGQLGGM | Carnobacterium divergens |
| | LAGALGGPGGVVLGGIGGAI | [Worobo et al., Journal of |
| | AGGCFN (SEQ ID NO: 7) | Bacteriology, 177, 3143-3149 |
| | | (1995)] |
| Garvicin A | IGGALGNALNGLGTWANMMNGG | Lactococcus garvieae |
| | GFVNQWQVYANKGKINQYRPY | [Maldonado-Barragan et al., |
| | (SEQ ID NO: 8) | Applied and Environmental |
| | | Microbiology, 79, 4336 (2013)] |
| Plantaricin E | FNRGGYNFGKSVRHVVDAIGSVA | LactoBacillus plantarum |
| | GIRGILKSIR (SEQ ID NO: 9) | [Diep et al., Journal of |
| | | Bacteriology, 178, 4472-4483 |
| | | (1996)] |
| Plantaricin F | VFHAYSARGVRNNYKSAVGPADW | LactoBacillus plantarum |
| | VISAVRGFIHG (SEQ ID NO: 10) | [Diep et al., supra] |
| Plantaricin J | GAWKNFWSSLRKGFYDGEAGRAI | LactoBacillus plantarum |
| | RR (SEQ ID NO: 11) | [Diep et al., supra] |
| Plantaricin K | RRSRKNGIGYAIGYAFGAVERAVL | LactoBacillus plantarum |
| | GGSRDYNK (SEQ ID NO: 12) | [Diep et al., supra] |

Examples of antimicrobial peptides also include those that are essentially identical to any one of the antimicrobial peptides in Table 4. As used herein, in the context of a protein “essentially identical” refers to a protein that differs from one of the proteins disclosed herein. A protein that is essentially identical to an antimicrobial peptide differs from one of the antimicrobial peptides in Table 4 at 1, 2, 3, 4, 5, 6, 7, 8, 9, 10, 11, or 12 amino acid residues and has antimicrobial activity. The difference can be a conservative substitution.

“Bacteriocins” refer to ribosomally synthesized antimicrobial peptides (AMPs) derived from bacterial species. Most bacteriocins have a very narrow target spectrum; individual bacteriocins are active against a just few species or genera. On the contrary, eukaryotic AMPs as well as traditional antibiotics are generally much less specific, targeting a large diversity of different bacteria. Consequently, in terms of potency and specificity, bacteriocins may be superior to traditional antibiotics and eukaryotic AMPs.

Bacteriocins can thus be very useful in therapeutic treatments where a particular pathogen is to be removed from a complex multi-species environment (such as in the gut) without causing adverse secondary effects as normally occur with common antibiotics.

The classification of bacteriocins has been under debate for a number of years. Herein, when discussing bacteriocins derived from Gram-positive bacteria, the classification scheme of Nes et al. [Food Science and Biotechnology, 675-690 (2007)] is followed. When discussing bacteriocins derived from Gram-negative bacteria, the classification scheme of Duquesne et al. [Duquesne et al., Natural Product Reports, 24, 708-734 (2007)] is followed.

Class II bacteriocins are provided and exemplified herein. The class II bacteriocins from Gram-negative bacteria include the class IIa and class IIb bacteriocins. Class IIa and class IIb bacteriocins from Gram-negative species employ homologous secretion systems and typically require fewer post-translational modifications compared to class I bacteriocins.

Examples of class II bacteriocins in Gram-negative bacteria include, but are not limited to, microcin V, microcin L, microcin N, microcin E492, microcin H47, microcin I, and microcin M.

The class II bacteriocins from Gram-positive bacteria again typically require few if any post-translational modifications compared to class I bacteriocins from Gram-positive bacteria. Class II Gram-positive bacteriocins are subdivided into five subclasses. The class IIa bacteriocins (pediocin-like bacteriocins) are the largest subgroup and contain an N-terminal consensus sequence-Tyr-Gly-Asn-Gly-Val-Xaa-Cys (SEQ ID NO: 29) across this group. The class IIb bacteriocins (two-peptide bacteriocins) require two different peptides for activity. Class IIc encompasses cyclic peptides, in which the N-terminal and C-terminal regions are covalently linked. Class IId cover single-peptide bacteriocins, which are not post-translationally modified and do not show the pediocin-like signature. Class IIe encompass those bacteriocins composed of three or four non-pediocin like peptides.

Class IIa bacteriocins are small (usually 37 to 48 amino acid), heat-stable, and non-post-translationally modified proteins that are typically positively charged and may contain an N-terminal consensus sequence -Tyr-Gly-Asn-Gly-(Val/Lys)-Xaa-Cys-(SEQ ID NO: 30). Another example of class II bacteriocins includes members of the subclass IIb bacteriocins. Class IIb bacteriocins are heterodimeric bacteriocins that require two different molecules often at approximately equal concentrations to exhibit optimal activity. Class IId bacteriocins lack a secretion tag. Class IIe are formed by degradation of a larger protein. Class IIc encompass the “other” class II bacteriocins that resemble class II bacteriocins but do not fall under the other subdivisions. Examples of class II bacteriocins include, but are not limited to, those shown in Table 4 above.

The sequence of a polynucleotide encoding an antimicrobial peptide can be easily predicted based on the standard genetic code. When an antimicrobial peptide is to be expressed in a particular microbe, a polynucleotide encoding the antimicrobial peptide can be produced with reference to preferred codon usage for the particular microbe.

Recombinant live bacterium provided herein can express and secrete one or more antimicrobial peptides.

### Secretion Tags

An exogenous polynucleotide encoding an antimicrobial peptide can include nucleotides encoding a secretion signaling protein, such that the antimicrobial peptide and the secretion signaling protein are fused and expressed as a single protein. A secretion signaling protein, or secretion tag, targets a protein for secretion out of the cell, and is usually present at the amino-terminal end of a protein. Secretion signaling proteins useful in prokaryotic microbes are known in the art and routinely used.

As used herein, “secretion tag” or “secretion signaling proteins” are protein sequences that are directly upstream of the mature protein that would be secreted out of the cell. For reviews on secretion and the use of secretion tags for heterologous gene expression in Bacillus, refer to Fu et al. [Biotechnology Advances, 25, 1-12 (2007)] and Simonen and Palva [Microbiological Reviews, 57, 109-137 (1993)].

The secretion tag can be derived from Bacillus spp, such as Bacillus subtilis, Bacillus licheniformis, Bacillus paralicheniformis, Bacillus amyliquofaciens, Bacillus coagulins. The secretion tag can be a sec-dependent or tat-dependent secretion tag. The sec-dependent secretion tag can be the AmyQ secretion tag, the LipA secretion tag, the Mdr secretion tag, the AprE secretion tag, the AbnB secretion tag, the EgIS secretion tag, the PhrC secretion tag or the LipA secretion tag. The tat-dependent secretion tag can be the PhoD secretion tag or the YwbN (EfeB) secretion tag. The secretion tag can be a variant of the aforenoted secretion tags.

The Sec and Tat secretion pathways are common routes for secretion from Bacillus spp. The regions of a secretion tag that uses the Sec pathway include a positively charged NH2 terminus followed by a hydrophobic region in the middle termed the H region and ends with a polar C region where the cleavage site is located. The consensus cleavage motif for a Sec-dependent secretion tag is typically A-X-A or V-X-A but this is not always the case. The secretion tags can serve many purposes such as preventing premature folding of protein and the degradation of the protein in the cell. Most importantly the secretion tags guide the protein to the correct secretion machinery at the membrane. During or directly after translocation, designated type I signal peptidases (SPase) cut the mature protein away from the secretion tag at the cleavage site. After the successful cleavage of the secretion tag from the mature peptide, the tag is subsequently degraded by SppA and TepA because accumulation of signal peptides can inhibit secretion.

After the precursor protein is ribosomally-synthesized, a signal recognition particle (SRP) forms a ribonucleoprotein complex with the signal peptide. The SRP consists of a small condition RNA (scRNA) with a Ffh (GTPase) protein and two molecules of the Hbsu protein. After a successful interaction, the SRP guides the complex to a membrane protein called FtsY where precursor protein is then passed along to the Sec translocases. The motor component of the translocation machinery for the Sec-dependent pathway is the SecA protein providing the energy via ATP hydrolysis domain for the translocation. The SecA is a transmembrane protein essential for targeting and translocation the desired secreted protein. The SecY, SecE, SecG, and SecDF proteins are integral proteins involved in the translocation of the unfolded mature target proteins across the membrane. In addition to the translocation, there are also several chaperone proteins located in the cytoplasm and extracellular space that aid in protein folding and ensure there is minimal aggregation of protein in the cell.

Bacillus spp. are favorable for production of heterologous protein due to absence of an outer membrane which would theoretically simplify translocation and secretion. Additionally, Bacillus spp. are known to efficiently secrete a large number of proteins, the majority of which use a general secretion pathway.

Despite their natural propensity for protein secretion, there are a number of hurdles to overcome to successfully secrete heterologous proteins from Bacillus spp. One of the primary challenges encountered is protease activity. Bacillus spp. often secrete large quantities of proteases to break down proteins in the environment. If a target protein is small and linear it can be easily degraded at a higher rate than it can be produced. Other issues that can arise could be interactions with the secretion tag and target proteins and interactions with the secretion machinery.

Table 5 shows the different exemplary Sec and Tat-dependent secretion tags that were fused to mature peptides as described in the Examples. Included with each secretion tag is the amino acid sequence that ends with the cleavage site where the tag is cleaved from the mature peptide. The cleavage site is generally recognized by the A-X-A or V-X-A motif (italicized) though this is not the case for every Sec-dependent secretion tag. Also listed is the function of the protein that the secretion tag is originally attached to in B. subtilis 168's genome (exception is AmyQ which is found in B. amyloliquefacien's genome).

TABLE 5
Examples of Sec and Tat-dependent secretion tags for Bacillus spp.

| Secretion | | |
|---|---|---|
| Tag | Amino Acid Sequence | Wild-Type Protein Function |
| AmyQ | MIQKRKRTVSFRLVLMCTLLFVSLPITKTSA (SEQ ID | Amylase |
| | NO: 13) | (B. amyloliquefaciens) |
| *LipA | MKFVKRRIIALVTILMLSVTSLFALQPSAKAA (SEQ ID | Alkaliphilic lipase |
| | NO: 14) | |
| Mdr | MDTTTAKQASTKFVVLGLLLGILMSAMDNTIVATA | Multi-drug efflux transporter |
| | (SEQ ID NO: 15) | |
| AprE | MRSKKLWISLLFALTLIFTMAFSNMSAQA (SEQ ID | Serine alkaline protease |
| | NO: 16) | (Ca2+-dependent subtilisin |
| | | E) |
| PhrC | MKLKSKLFVICLAAAAIFTAAGVSANA (SEQ ID NO: | secreted regulator of the |
| | 17) | activity of phosphatase |
| | | RapC and competence and |
| | | sporulation stimulating |
| | | factor (CSF) |
| AbnA | MKKKKTWKRFLHFSSAALAAGLIFTSAAPAEA (SEQ | arabinan-endo-1,5-alpha-L- |
| | ID NO: 18) | arabinase |
| AbnB | MFNRLFRVCFLAALIMAFTLPNSVYA (SEQ ID NO: | arabinan-endo-1,5-alpha-L- |
| | 19) | arabinase (Ca2+- |
| | | dependent) |
| EglS | MKRSISIFITCLLITLLTMGGMIASPASA (SEQ ID NO: | endo-1,4-beta-glucanase |
| | 20) | |
| PhoD | MAYDSRFDEWVQKLKEESFQNNTFDRRKFIQGAGKI | alkaline phosphatase D |
| | AGLSLGLTIAQSASA (SEQ ID NO: 21) | |
| YwbN | MSDEQKKPEQIHRRDILKWGAMAGAAVAIGASGLG | peroxidase |
| (EfeB) | GLAPLVASA (SEQ ID NO: 22) | |

The secretion tags derived from Bacillus subtilis such as Bacillus 168 can be inserted upstream of an antimicrobial peptide gene fragment to generate a secretion tag library, e.g., as described in Example 11.

The secretion tag library with the antimicrobial peptide is transformed into a desired Bacillus spp. isolate, e.g., as described in Example 9.

Genetic Manipulation of Recombinant Bacillus Spp. Strains

Bacillus spp. provided herein are made competent and transformed with an engineered plasmid, e.g., as described in Example 9.

Bacillus spp. provided can be made rifampicin resistant, e.g., as described in Example 12.

An essential gene (ex. dapA or thyA) can be knocked out of the genome of provided Bacillus spp. yielding an auxotroph, e.g., as described in Example 13.

Exemplary recombinant Bacillus isolates GP01270, GP01284, GP01324, GP01416-GP01418, and GP01434-GP01439 are provided herein.

### Antimicrobial Activity Against Indicator Bacterial Strains

As used herein, an “indicator strain” is a strain used to evaluate the production of the peptide of interest. Examples of suitable indicator strains include but are not limited to those listed in Table 6 below. The indicator strain can be a member of the genus Enterococcus, such as E. faecalis and E. faecium. Methods for testing the activity of an antimicrobial peptide include, but are not limited to, the stab-on-agar test as well as other methods useful for evaluating the activity of bacteriocins. Such methods are known in the art and are routine.

TABLE 6
Exemplary Indicator Strains
Indicator Strains

Enterococcus faecium 8E-9
Listeria monocytogenes CDC 7762
Clostridium perfringens SPRG-6
Clostridium perfringens 26
Clostridium perfringens 39
Clostridium perfringens NAH-JP1011

The recombinant live Bacillus spp. can be tested against Enterococcus faecium embedded in BHI agar. In this assay, the level of antimicrobial activity correlates to the diameter of the zone of inhibition, e.g., as described in Example 14.

The recombinant live Bacillus spp. can be tested against Listeria monocytogenes embedded in BHI agar. In this assay, the level of antimicrobial activity correlates with the diameter of the zone of inhibition, e.g., as described in Example 14.

The recombinant live Bacillus spp. can be tested against various Clostridium perfringens strains using agar diffusion assays. In this assay, recombinant Bacillus spp. are selected for large zones of inhibition on Clostridia perfringens plates, e.g., as described in Example 15.

The minimum inhibitory concentration (MIC) of the recombinant live Bacillus spp. supernatant can be measured against Enterococcus faecium. Bacillus spp. with supernatant exhibiting the lowest MIC against E. faecium are selected, e.g., as described in Example 14.

The minimum inhibitory concentration (MIC) of the recombinant live Bacillus spp. supernatant can be measured against Listeria monocytogenes. Bacillus spp. with supernatant exhibiting the lowest MIC against L. monocytogenes are selected, e.g., as described in Example 14.

The minimum inhibitory concentration (MIC) of the recombinant live Bacillus spp. supernatant can be measured against various Clostridium perfringens strains. Bacillus spp. with supernatant exhibiting the lowest MIC against various C. perfringens strains are selected.

Recombinant live Bacillus spp. secretion tag library isolates can be tested against Enterococcus faecium embedded in a plate with BHI agar. In this assay, the level of antimicrobial activity correlates to the diameter of the zone of inhibition on the plate. Bacillus spp. are selected for a large zone of inhibition diameter, e.g., as described in Example 11.

### In Vitro Assays Mimicking the Intestinal Tract

Survival of recombinant or unmodified Bacillus spp. can be tested in intestinal tract-mimicking environments (ex. low pH, presence of bile salts); or in intestinal contents (ex. stomach/gizzard contents, duodenum contents, jejunum contents, ileum contents, large intestine contents, ceca contents). Growth of recombinant or unmodified Bacillus spp. are tested in intestinal tract-mimicking environments (ex. minimal medium, rich medium) as described in Example 16; or in intestinal contents (ex. stomach/gizzard contents, duodenum contents, jejunum contents, ileum contents, large intestine contents, ceca contents, mucus from various parts, or combinations of intestinal contents and laboratory-derived medium), e.g., as described in Example 16.

### Production and Formulation of Recombinant Bacillus Spp.

Recombinant live Bacillus spp. of the disclosure can be produced by a batch or fed-batch fermentation process. The recombinant Bacillus spp. can be harvested out of fermentation broth via centrifugation.

Recombinant live Bacillus spp. of the disclosure can be in a cell paste that is blended with excipients and spray dried to powder form. The excipients can include, for example, one or more of trehalose, maltodextrin, sucrose, whey protein and calcium carbonate.

Recombinant live Bacillus spp. in a dry powder can be mixed with animal feed and pelletized. Alternatively or in addition, recombinant Bacillus spp. in a dry powder can be added to animal farmhouse water supply in a 1:128 dilution via a medicator pump.

In Vivo Use Against C. perfringens

Methods are provided herein to treat disease conditions caused by C perfringens (such as necrotic enteritis) in animals.

Disease conditions caused by C perfringens include, but are not limited to, those in Table 7 below.

TABLE 7
Exemplary disease conditions caused by C perfringens

| C. | | |
|---|---|---|
| perfringens | Animal species | |
| Type | affected | Disease condition |
| A | Avian | Poultry necrotic enteritis, notably broiler chickens |
| | Horses, dogs | Foal necrotizing enteritis; canine haemorrhagic gastroenteritis. |
| | Cattle, sheep | Clostridial abomasitis; may be caused by several pathogenic |
| | | enteric clostridial species |
| | Cattle | Hemorrhagic enteritis (“enterotoxaemia”) of calves, especially |
| | | Belgian Blue; |
| | Pigs | Neonatal mild necrotizing enterocolitis of piglets. |
| | Sheep | Yellow lamb disease |
| B | Sheep | Lamb dysentery; chronic enteritis (“pine”) in older lambs. |
| C | Cows, goats, | Hemorrhagic and/or necrotizing enteritis neonatal farm |
| | horses, humans, | animals (calves, foals, goat kids, lambs, piglets), and others; |
| | pigs, sheep | humans (“pigbel”, “darmbrand”). |
| D | Goats, sheep, | Enterotoxaemia (“pulpy kidney disease”) of sheep and goats; |
| | cows | enteritis adult goats; enterotoxaemia in adult cattle. |
| F | Cows, rabbits, | Haemorrhagic enteritis in calves and possibly lambs; enteritis |
| | sheep | in rabbits |

In exemplary prophylactic treatment methods herein, recombinant live Bacillus spp. of the disclosure are fed to animals (e.g., fed as described in Example 17) to protect the animals against a C. perfringens strain or strains. In exemplary therapeutic treatment methods herein, recombinant Bacillus spp. of the disclosure are fed to animals (e.g., as described in Example 17) infected with a C. perfringens strain or strains. An effective amount of bacterium fed is an amount that prevents development of a disease condition, that alleviates (eliminates or reduces) at least one symptom associated with the disease condition being treated, that slows or prevent progression of the disease condition, that diminishes the extent of the disease condition, that results in remission (partial or total) of the disease condition, and/or that prolongs survival. Fed animals (such as chickens) exhibit, for example, one or more of: reduced gross lesion scores, reduced morbidity, reduced mortality; and/or one or both of increased feed conversion ratio and average daily gain.

### General Terms

As used herein, the term “protein” or refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, trimers, tetramers). Thus, the terms peptide, oligopeptide, enzyme, subunit, and protein are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a plasmid or vector, a portion of a plasmid or vector. A polynucleotide may include nucleotide sequences having different functions, including, for instance, coding regions, and non-coding regions such as regulatory regions.

As used herein, the terms “coding region,” “coding sequence,” and “open reading frame” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, ribosome binding sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

As used herein, a “polycistronic mRNA” refers to a transcription product that includes two or more coding regions. Expression of the two or more coding regions is controlled by a single promoter, and the series of the two or more coding regions that are transcribed to produce a polycistronic mRNA is referred to as an operon.

As used herein, “genetically modified bacterium” or “recombinant bacterium” refers to a bacterium which has been altered “by the hand of man.” A genetically modified bacterium or recombinant bacterium includes a bacterium into which has been introduced an exogenous polynucleotide, e.g., an expression vector.

As used herein, a “vector” is a nucleic acid (e.g., DNA) used as a vehicle to artificially carry genetic material (e.g., an engineered nucleic acid) into a cell where, for example, the nucleic acid can be replicated and/or expressed. A non-limiting example of a vector is a plasmid. Plasmids are considered vectors herein and are double-stranded generally circular DNA molecules that are capable of automatically replicating in a host cell. Plasmids typically contain an origin of replication that allows for semi-independent replication of the plasmid in the host and also the transgene insert.

Plasmids may have more features, including, for example, a “multiple cloning site,” which includes nucleotide overhangs for insertion of a nucleic acid insert, and multiple restriction enzyme consensus sites to either side of the insert.

As used herein, an “exogenous protein” and “exogenous polynucleotide” refer to a protein and polynucleotide, respectively, which are not normally or naturally found in a microbe, and/or has been introduced into a microbe. An exogenous polynucleotide may be separate from the genomic DNA of a cell (e.g., it may be a vector, such as a plasmid), or an exogenous polynucleotide may be integrated into the genomic DNA of a cell.

As used herein, a “heterologous” polynucleotide, such as a heterologous promoter, refers to a polynucleotide that is not normally or naturally found in nature operably linked to another polynucleotide, such as a coding region. As used herein, a “heterologous” protein or “heterologous” amino acids refers to amino acids that are not normally or naturally found in nature flanking an amino acid sequence.

As used herein, the term “variant” refers to a polypeptide that comprises one or more differences in the amino acid sequence of the variant relative to a reference sequence. For example, a “variant” polypeptide may include one or more deletions, additions or substitutions relative to a reference sequence. The term “variant” is not intended to limit the variant polypeptide to only those polypeptides made by the modification of an existing polypeptide or nucleic acid molecule encoding the reference sequence, but may include variant polypeptides that are made de novo or starting from a polypeptide other than the reference sequence. The term “variant” is also used in the contest of a polynucleotide that comprises one or more differences in the nucleotide sequence of the variant relative to a reference sequence.

As used herein, the term “conservative variant” shall refer to sequences which reflect the incorporation of conservative amino acid substitutions. Conservative substitution tables are well known in the art. Conservative amino acid substitutions are defined to result from exchange of amino acids residues from within one of the following classes of residues: Class 1: Ala, Gly, Ser, Thr, and Pro (representing small aliphatic side chains and hydroxyl group side chains); Class 2: Cys, Ser, Thr, and Tyr (representing side chains including an —OH or —SH group); Class 3: Glu, Asp, Asn, and Gln (carboxyl group containing side chains): Class 4: His, Arg, and Lys (representing basic side chains); Class 5: Ile, Val, Leu, Phe, and Met (representing hydrophobic side chains); and Class 6: Phe, Trp, Tyr, and His (representing aromatic side chains).

As used herein, a protein may be “structurally similar” to a reference protein if the amino acid sequence of the protein possesses a specified amount of sequence similarity and/or sequence identity compared to the reference protein. Thus, a protein may be “structurally similar” to a reference protein if, compared to the reference protein, it possesses a sufficient level of amino acid sequence identity, amino acid sequence similarity, or a combination thereof.

Structural similarity of two proteins can be determined by aligning the residues of the two proteins (for example, a candidate protein and any appropriate reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. A candidate protein is the protein being compared to the reference protein. A candidate protein may be isolated, for example, from a microbe, or can be produced using recombinant techniques, or chemically or enzymatically synthesized.

Unless modified as otherwise described herein, a pair-wise comparison analysis of amino acid sequences can be carried out using the Blastp program of the BLAST 2 search algorithm, as available on the National Center for Biotechnology Information (NCBI) website [Tatusova and Madden, FEMS Microbiology Letters 174, 247-250 (1999)]. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions.

Thus, as used herein, a candidate protein useful in the methods and compositions described herein includes those with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate protein useful in the methods described herein includes those with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Conditions that are “suitable” for an event to occur, such as expression of an exogenous polynucleotide in a cell to produce a protein, or production of a product, or “suitable” conditions are conditions that do not prevent such events from occurring. Thus, these conditions permit, enhance, facilitate, and/or are conducive to the event.

As used herein, an “animal” includes, but is not limited to, members of the class Mammalia and members of the class Aves, such as human, avian, bovine, caprine, ovine, porcine, equine, canine, and feline.

As used herein, the recitations of numerical ranges by endpoints include all numbers subsumed within that range (e.g., 1 to 5 includes 1, 1.5, 2, 2.75, 3, 3.80, 4, 5, etc.).

As used herein, “probiotics” are “live microorganisms that, when administered in adequate amounts, confer a health benefit on the host”, as defined by The Food and Agriculture Organization of the United Nations (FAO).

As used herein, “antimicrobial live therapeutics” or “cellbots”, or “recombinant live Bacillus spp.” are Bacillus spp. probiotics that are modified using recombinant biology techniques to express and deliver antimicrobial proteins/peptides (including, but not limited to, bacteriocins).

As used herein, “antimicrobial peptides” (sometimes referred to herein as AMPs) are small proteins, typically between about 10 and about 100 amino acids in length that inhibit, and often kill, certain bacteria. As such, an antimicrobial peptide has antimicrobial activity that inhibits or kills a target microbe.

The “target microbe” may be a Gram-positive bacterium that is member of the genus Clostridia. Examples of Clostridia include, for instance, Clostridia perfringens and Clostridia difficile.

The “target microbe” may be in vitro or in vivo. For instance, in one embodiment, a target microbe may be one that is present in the gastrointestinal tract or urogenital system of a subject, and optionally may be pathogenic to the subject. For instance, in another embodiment, a target microbe may be one that is present in the ovaries of hens, contaminating the eggs inside the chicken before the shells are formed.

As used herein, the singular forms “a,” “an,” and “the” include plural referents unless the content clearly dictates otherwise. Thus, for example, reference to a composition containing “a bacterium” includes a composition with more than one bacterium. It should also be noted that the term “or” is generally employed in its sense including “and/or” unless the content clearly dictates otherwise.

As used herein, the word “engineered” describes a system, apparatus, or other structure that is constructed or configured to perform a particular task or adopt a particular configuration. The phrase “engineered” can be used interchangeably with other similar phrases such as arranged and configured, constructed and arranged, constructed, manufactured and arranged, and the like.

For any method disclosed herein that includes discrete steps, the steps may be conducted in any feasible order. And, as appropriate, any combination of two or more steps may be conducted simultaneously.

Embodiments described herein are not to be taken in isolation from the rest of the disclosure and, as such, can be combined with other features or embodiments as may be appropriate based on the general knowledge and understanding of a skilled reader.

As used herein, “may,” “may comprise,” “may be,” “can,” “can comprise” and “can be” all indicate something envisaged by the inventors that is functional and available as part of the subject matter provided.

All publications and patent applications referred to herein are incorporated by reference to the same extent as if each individual publication or patent application was specifically and individually indicated as being incorporated by reference.

Aspects and advantages of the disclosure may be better understood with reference to the following examples. These examples are intended to be representative and are not intended to limit the overall scope of the disclosure herein.

### EXAMPLES

Various aspects and advantages of the disclosure are illustrated by the following non-limiting examples.

### Example 1: Isolation of Bacillus Spp. From Chicken Intestines

The intestines of sacrificed healthy chickens and those infected with Clostridium perfringens were excised and the jejunum, ileum, and cecum sections were separated. The sections were stored in the −20° C. freezer until use. The sections were then thawed at room temperature and the intestinal contents of each section were isolated and placed into tubes.

Various techniques were used to isolate the Bacillus spp. spores which included boiling, ethanol shock, or a combination of both. The procedure for boiling included diluting the intestinal sample in phosphate buffered saline at a 1:1 ratio followed by heating at 80° C. for 20 minutes. The procedure for ethanol shock included mixing the intestinal contents with 95% ethanol at a 1:1 ratio and incubating at room temperature for 1 hour. If both selection methods were used, the ethanol shock was done first followed by the boiling.

After the selection process was completed, ~0.5 mL of the treated intestinal contents was plated on various microbiological media such as Brain Heart Infusion agar, Luria-Bertani agar, Mueller-Hinton agar, and Nutrient Agar. Plates were incubated overnight at 37° C. and individual colonies were isolated and transferred for subsequent testing and preservation.

### Example 2: DNA Fingerprinting to Establish Isolate Uniqueness

Prospective Bacillus spp. isolates that were morphologically distinct from one another were selected for DNA fingerprinting. In this procedure, a polymerase chain reaction was performed on the Bacillus colonies using BOX A1R primer (5′-CTACGGCAAGGCGACGCTGACG-3′, SEQ ID NO: 31). The PCR conditions are described below. After the PCR was complete, the reactions were loaded onto a 1.5% agarose gel and run at 120V for two hours. Isolates that had similar band patterns were grouped together and a representative isolates from each group were selected for subsequent testing and preservation.

The PCR was performed using Promega GoTaq Green Mastermix according to the manufacturer's protocol. The PCR reaction mixture consisted of 12.5 uL of GoTaq Green mastermix, 2 uL of the BOXA1R primer (10 UM solution), and 10.5 μL of autoclaved distilled water. A colony of Bacillus was swabbed with a sterile tip and placed into the reaction mixtures. The tip was then ground into the reaction tube to dislodge cells and release their DNA. The thermalcycler settings were as follows:
Initial denaturation 95° C. for 10 minutes
35 cycles
95° C. for 30 seconds
50° C. for 30 seconds
72° C. for 8 minutes
Final extension 72° C. for 5 min

### Example 3: 16S Sequencing of the Bacillus Spp. Isolates

Bacillus spp. isolates were sent for 16S rRNA sequencing to identify the species of the isolate. The 16S rRNA gene was amplified using polymerase chain reaction (PCR) with the universal primers, 27F (5′AGAGTTTGATCMTGGCTCAG-3′, SEQ ID NO: 32) and 1492R (5′-GGYTACCTTGTTACGACTT-3′, SEQ ID NO: 33). The reaction conditions are stated below. PCR reactions were visualized using gel electrophoresis and the expected band (located at ~1500 basepairs) was excised for each strain, gel-purified, and sent for Sanger sequencing through AGCT Inc. Species was estimated via standard sequence alignment of the resulting 16S rRNA sequences using the Basic Local Alignment Search Tool [Nucleotide BLAST (blast.ncbi.nlm.nih.gov/Blast)]. Through 16S sequencing GP01336 was initially identified as Bacillus licheniformis and GP01252 was identified as Bacillus oleronius. GP01336 (ATCC Accession No. PTA-127307) was later discovered to a Bacillus paralicheniformis strain after the whole genome was sequenced.

The PCR was performed using Promega GoTaq Green Mastermix. The PCR reaction mixture consisted of 12.5 uL of GoTaq Green mastermix, 1 uL of each primer (10 μM solution), and 10.5 uL of autoclaved distilled water. A colony of Bacillus was swabbed with a sterile tip and placed into the reaction mixtures. The tip was then ground into the reaction tube to dislodge cells and release their DNA. The thermalcycler settings were as follows:
Initial denaturation 98° C. for 10 minutes
35 cycles
98° C. for 30 seconds
55° C. for 30 seconds
72° C. for 1 minute 30 seconds
Final extension 72° C. for 10 min

### Example 4: Protease Activity of Bacillus Spp. Against Antimicrobial Peptides

Several attempts were made to express and secrete various class II bacteriocins from Bacillus subtilis 168 without success. Standard troubleshooting with genetic manipulation suggested that the lack of production was not due to transcriptional or translational bottlenecks. Additionally, several secretion mechanisms were attempted.

To test whether the strain was producing but rapidly inactivating the peptides of interest, cultures of Bacillus subtilis 168 were grown overnight and cells were removed by centrifugation. The resulting supernatant was then sterilized via filtration through a 0.22 um polyethersulfone filter.

B. subtilis supernatant was then incubated with the peptides of interest and peptide potency was evaluated using a minimum inhibitory concentration assay (see Example 14). It was found that incubation with B. subtilis 168 supernatant for just 3 hours resulted in complete elimination of peptide activity.

Protease activity has been examined in the context of heterologous protein expression [Wu et al., Applied and Environmental Microbiology, 68, 3261-3269 (2002)]. Suspected proteases in the genome of the producer strain have been knocked out, as in Thompson et al., FEMS Microbiology Letters, 367, 162 (2020) which describes the 8-gene knockout Bacillus subtilis Δ8-22. Strains with these genes knocked out have exhibited higher yields of other heterologous proteins [Wu et al., supra].

B. subtilis Δ8-22 was tested with several genetic constructs to produce class II bacteriocins. However, no production was observed. Supernatant protease activity assays were performed with this strain. Despite the elimination of all detectable protease genes, rapid peptide inactivation was still observed. Thus, the heterologous production of class II bacteriocins was unable to be attained using traditional methods previously known to those skilled in the art.

A solution contemplated herein is that the production strain is selected through medium-throughput screening of natural isolates for compatibility with the peptide of interest. To determine compatibility, supernatant protease assays were performed for panels of Bacillus isolates and isolates exhibiting the lowest levels of protease activity or peptide inactivation were selected for genetic manipulation.

For the protease assay, selected isolates were inoculated into Brain Heart Infusion (BHI) medium and grown shaking at 37° C. for ~20 hr. After the incubation, 2 mL of each Bacillus culture was transferred to 2 ml conical tubes and centrifuged to remove cells (13,000×g, 1 minute). Bacillus spp. supernatant was then filter-sterilized using a 0.22 um polyethersulfone filter.

The peptide of interest (for example, enterocin B, hiracin JM79, enterocin A) was produced using various strains of bacteria known to produce high quantities of the peptides. 50 mL cultures of the producer strains were grown in BHI at 37° C. for ~20 hr. Cultures were then centrifuged to remove cells and supernatant was sterilized by boiling for 10 minutes at 100° C. The act of boiling at this time and temperature has been found to eliminate any protease activity from the producer supernatant without impacting peptide activity. This supernatant will be referred to as the “peptide stock.”

500 uL of Bacillus supernatant was mixed 500 uL of the peptide stock and incubated at 37° C. for 3 hours. 500 uL of plain BHI was mixed with 500 uL of the peptide stock as a negative control for protease activity. 500 uL of plain B. subtilis 168 supernatant was mixed with 500 uL of peptide stock as a positive control for protease activity. After the set time of incubation, 180 uL of the mixed supernatant was placed into sterile PCR tubes and boiled in the thermocycler (100° C., 10 min) to inactivate protease activity.

For the MIC assay, 120 uL of each of the treated supernatants was placed into the wells of a 96 well plate and serial 2× dilutions were performed using phosphate buffered saline (PBS). An overnight culture of indicator strain (ex. E. faecium or Listeria monocytogenes) was then diluted in fresh growth medium (BHI) to a concentration of ~1000 CFU/mL. 240 uL of diluted indicator strain was then added to each well. The plates were incubated overnight at 37° C.

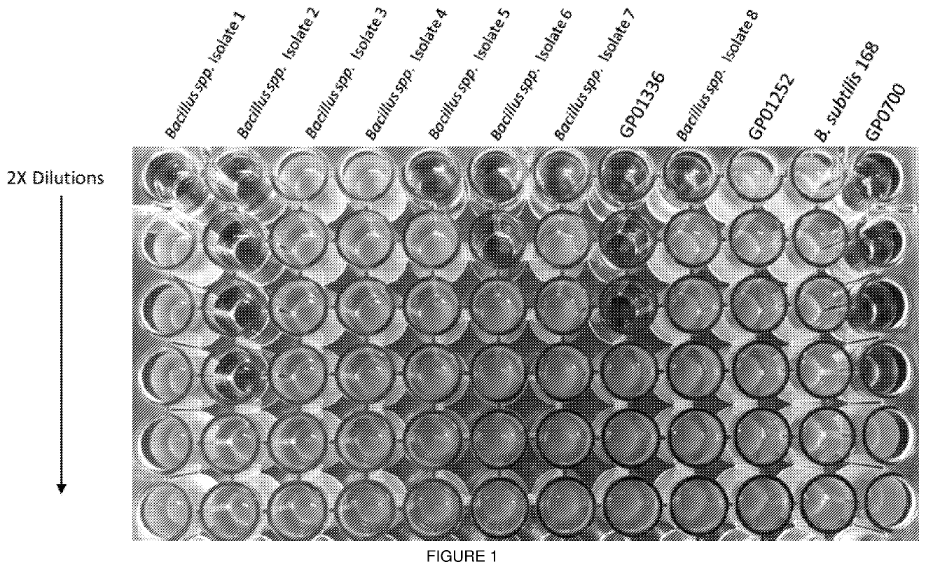

Approximately 18 hours post-incubation, the plates were examined. FIG. 1 shows an example of a protease assay for ten Bacillus isolates. Peptide potency of the samples was determined based on the minimum amount of sample required to attain full inhibition of the indicator strain (the MIC). Thus, samples which inhibited at the highest dilutions were considered to contain the most peptide of interest and thus the lowest levels of peptide degradation.

A Bacillus isolate was considered to exhibit “favorable protease activity” if inhibition of the indicator strain could be observed at any dilution of that strain's peptide/supernatant mixture. If no inhibition could be observed, the isolate was considered unfavorable and was discounted for further assessment.

### Example 5: Testing Isolates Innate Antimicrobial Activity Against Indicator Strains

Bacillus isolate supernatants in the absence of peptide were also tested against the indicator strain used in the protease activity. This was done for two purposes. The first intention was to ensure that inhibition observed from peptide/supernatant mixtures was due to the peptide and not due to antimicrobial activity of the Bacillus isolate supernatant. The second purpose was to identify if strains may carry their own antimicrobial activity.

For these assays, 60 μL of the filtered Bacillus spp. supernatant collected during the protease assays was boiled and placed into the wells of a 96 well plate. 2× dilutions were performed on supernatants using phosphate-buffered saline. An overnight culture of indicator strain (ex. E. faecium or Listeria monocytogenes) was then diluted in fresh growth medium (BHI) to a concentration of ~1000 CFU/mL. 270 uL of diluted indicator strain was then added to each well. The plates were incubated overnight at 37° C.

Plates were analyzed for growth after ~18 hours of incubation. Bacillus isolates which produced supernatants that resulted in inhibition of the indicator strain were noted.

In addition to the supernatant assays, agar diffusion assays were also performed using the Bacillus spp. strains. Indicator plates were made by mixing the indicator strain (ex. E. faecium 8E9 or Listeria monocytogenes CDC 7762) in warm BHI agar at a concentration of 0.5 uL overnight culture/mL agar or 0.3 μL overnight culture/mL agar, respectively and pouring the seeded agar into plates. Plates were left at room temperature for ~15-30 minutes to solidify.

Each Bacillus spp. strain was swabbed with a sterile pipet tip then stabbed into the plate. Plates were then incubated overnight at 37° C. The next day, the Bacillus spp. that produced zones of inhibition against a given strain were assumed to have innate antimicrobial activity against that given indicator strain. The presence of innate activity against indicator strain did not qualify or disqualify a certain Bacillus spp. isolate but was taken into consideration when assessing the peptide activity against these same indicator strains. Neither GP01252 or GP01336 had innate activity against 8E9 or CDC 7762.

### Example 6: Testing for Susceptibility of Bacillus Spp. Isolates to the Peptide of Interest

Various Bacillus spp. isolates were grown overnight in 3 mL BHI cultures. The next day, a sample of each of the cultures was inoculated into warmed BHI agar medium at various concentrations (1-3 μL overnight culture/mL agar) and poured into plates. After the plates dried, a sample of peptide producer strain (ex. an E. coli strain engineered to produce Enterocin B) was stabbed into the plates along with its unmodified counterpart. The plates were then incubated overnight at 37° C. The next day the plates were examined for production of zones of inhibition from the peptide-producing strain that were significantly different from the unmodified counterpart.

It is preferable that the Bacillus spp. isolate is not susceptible to the peptide of interest so that the immunity gene does not have to be included in the genetic construct. Absence of the immunity gene would alleviate fears of genetically transferring the gene in the ambient microbiome, particularly the target species. Bacillus spp. isolates that exhibited no susceptibility the peptide of interest were prioritized over those that were susceptible. GP01336 was not susceptible to enterocin A and GP01252 was slightly susceptible to the presence of enterocin A.

### Example 7: Testing for Antibiotic Resistance of the Bacillus Spp.

One of the primary concerns when considering any new probiotic is their resistance to antibiotics and their ability to transfer these resistance components to other microbes. All Bacillus isolates were screened for antibiotic resistance against a panel of antibiotics. Screening was done by first growing the isolates overnight on BHI agar. Isolates were then struck onto BHI agar containing the antibiotic of interest.

Examples of antibiotics tested and their concentrations are spectinomycin (100 ug/mL), kanamycin (20 ug/mL) and erythromycin (0.5 ug/mL).

Isolates were deemed favorable if they exhibited no observable growth on antibiotic plates.

### Example 8: Testing for Hemolytic Activity in Bacillus Spp.

GP01252 and GP01336 were tested for hemolytic activity by streaking fresh patches of each isolate on to Blood Agar medium (Thermo Scientific TSA with Sheep blood, part number R01201). Plates were incubated overnight at 37° C. The following morning, plates were analyzed for signs of beta-hemolysis which includes a clear halo containing lysed red blood cells surrounding the bacterial growth. GP01252 and GP01336 showed no signs of beta-hemolysis.

### Example 9: Transformation of Bacillus Spp. Isolates with Plasmids

Antimicrobial peptide plasmid design is described in detail in Example 10. GP01252 and GP01336 were transformed with the plasmid of interest via electroporation. Cells were made competent using the protocol described by Lu et al. [Letters in Applied Microbiology, 55, 9-14 (2012)]. This protocol was originally intended for transformation of Bacillus subtilis 168 derivatives but was found to be effective for various isolates of Bacillus.

### Making Electrocompetent Bacillus Stocks

GP01252 and GP01336 were struck out on lysogeny broth (LB) plates and incubated overnight at 37° C. The next day, a colony was selected from each of the plates and was inoculated into a 3 mL LB culture and incubated overnight shaking at 37° C. The following day, 50 uL of the overnight culture was inoculated into 3 mL of LB and incubated shaking at 37° C. for 17 hrs. After the 17 hr incubation, 1 mL of the culture was inoculated into a 100 mL Erlenmeyer flask containing 40 mL of LB+0.5 M sorbitol. The flask culture was incubated shaking at 37° C. until the 600 nm optical density (OD600) reached ~0.8.

Once the cultures had reached the correct OD600, the cultures were placed into 50 mL conical tubes and incubated on ice for 5 minutes. After the 5-minute incubation, the cultures were centrifuged at 5,000×g for 10 min at 4° C. to collect the cells. After the cells were pelleted, 15 mL of ice-cold Bacillus electroporation buffer (0.5M sorbitol, 0.5M trehalose, and 0.5M mannitol in 10% glycerol) was dispensed into the tubes and the pellets were resuspended via shaking. Two additional wash cycles were performed in which cells were centrifuged at 5000-6,000×g for 10-20 minutes then resuspended in 15 mL electroporation buffer. After the wash cycles, the remaining pellet was resuspended with 500 ul of the electroporation buffer solution. 80 uL aliquots were made and stored at −80° C. until use.

### Electroporation of Competent Cells

For the transformation of electrocompetent isolates, an 80 uL aliquot of competent GP01252 and GP01336 cells were thawed on ice and 100 ug of plasmid were added to the cells. The cells were transferred to a 1 mm cuvette and incubated on ice for 10 minutes. After the 10-minute incubation was complete, the cuvette containing the cells was placed in the electroporator. The settings were set at 2100V, 200 Ohms, and 25 uF and a single pulse was sent through the cells. The expected time constant was ~4.5 ms.

After a successful pulse, the cells were recovered in 1 mL of BHI containing 0.5M sorbitol and 0.38M mannitol shaking for 3 hours. After the 3 hour incubation, the cells were pelleted (5000×g, 5 minutes) and resuspended in ~100 uL of media. The cells were then plated on BHI plus kanamycin (20 ug/mL) and incubated overnight at 37° C.

Isolates that gave colonies the next day were tested for the presence of the plasmid via colony PCR and/or using agar diffusion assays (see Example 14). The size of the transformant colonies varied depending on the isolate and the smaller colonies tended to not grow well. This decrease in growth and viability may indicate incompatibility of the plasmid system in those isolates.

### Example 10: Generation of the Antimicrobial Peptide (AMP) Plasmid

Plasmids used to modify Bacillus for the production of AMPs contained at minimum, the following components: 1) a plasmid backbone consisting of a Bacillus-compatible origin of replication, an E. coli-compatible origin of replication, and a selection marker (either antibiotic-resistance or auxotrophy-dependent); 2) mature antimicrobial peptide fused to a secretion tag; 3) promoter region; 4) ribosomal binding site; and 5) terminator.

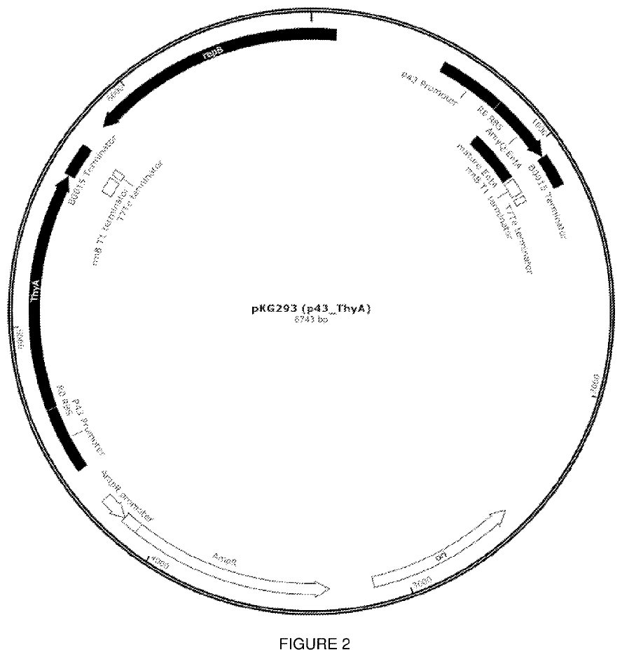

FIG. 2 depicts an example of a typical plasmid used to produce an antimicrobial peptide (in this case Enterocin A) from Bacillus isolates. Bacillus replication gene (repB) and the pUB110 origin of replication comprise the replication components necessary to maintain a high plasmid copy number in the Bacillus spp. cell. The plasmid contains the E. coli pMB1-dervied origin of replication necessary for cloning in E. coli. Additionally, an E. coli-compatible ampicillin resistance gene was included in preliminary plasmids to facilitate molecular cloning in E. coli. This resistance gene can be eliminated prior to the final application.

The plasmid in FIG. 2 also includes an example of the transcriptional unit for the antimicrobial peptide production as well as an example of the transcriptional unit for the auxotrophy-selection gene (thyA in this case). For the Bacillus spp. isolates that are not auxotrophs, a kanamycin resistance transcriptional unit is used in place of the thyA transcriptional unit.

To optimize peptide expression and isolate growth, numerous combinations of the above components were tested. Each component can impact the overall growth and production from the various isolates.

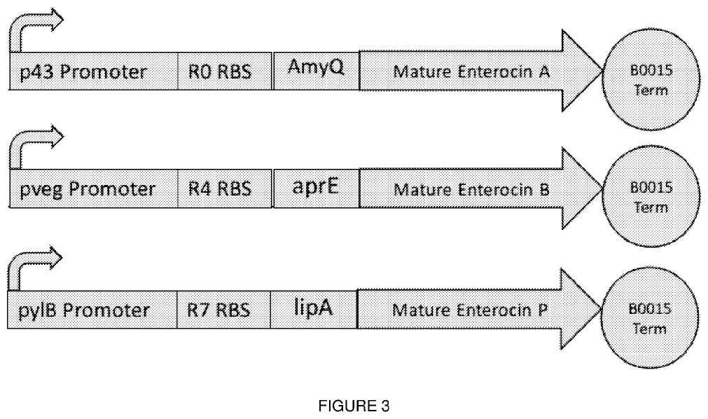

FIG. 3 shows examples of possible antimicrobial peptide transcriptional units. In one example, the promoter regulating the antimicrobial peptide transcriptional unit is the Bacillus-derived constitutive promoter, p43. In this example, the peptide of interest was the class IIa bacteriocin, enterocin A. Several promoters were tested with this construct including p43, pylB, and pveg (see Table 2 for additional promoters) using agar diffusion assays. See Example 14 for a description of the agar diffusion assay protocol). The enterocin A construct under the expression of the pylB promoter gave significantly smaller zones of inhibition compared to those containing the p43 promoter. It can thus be inferred that the pylB promoter does not operate well in the Bacillus spp. isolates described here.

The pveg promoter (a common strong constitutive promoter for Bacillus spp.) was also tested with this same enterocin A construct. With the enterocin A genetic construct and in the isolate tested, the pveg promoter was found to stunt the growth of the producer strain and was thus not favorable for peptide production.

FIG. 3 shows the use of the R0 ribosomal binding site and B0015 terminator sequence in the antimicrobial peptide transcriptional unit. These components, particularly the ribosomal binding site, provide additional critical parameters to tune antimicrobial peptide expression. For example, Guiziou et al., supra demonstrate that gene expression can be changed by orders of magnitude by altering the ribosomal binding site between those listed in Table 3. Similar trends were observed herein for antimicrobial peptide production in various Bacillus isolates.

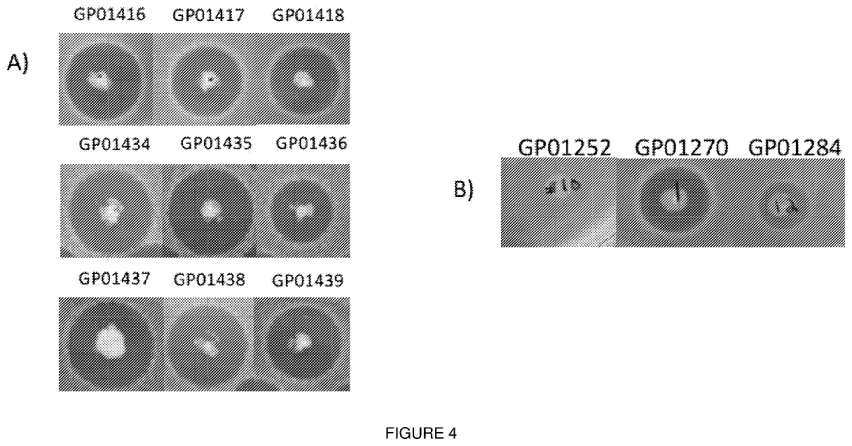

In addition to modifying the antimicrobial peptide transcriptional unit, it was necessary to find an appropriate expression system for the auxotrophic selection marker. In Example 13 we describe construction of the thyA-based system. FIG. 2 depicts the thyA gene under the control of the p43 promoter. However, it was found that growth, antimicrobial activity, and plasmid stability could be tuned by implementing different promoters and ribosomal binding sites (see FIG. 4).

### Example 11: Generation of a Bacillus Spp. Isolate Secretion Tag Library

The commercially-available pBE-S plasmid (Mobitec) was modified to enable a peptide library expression system compatible with a diverse panel of Bacillus isolates.

To generate the new plasmid, the mature peptide of interest (lacking the secretion tag) was amplified and inserted into the NdeI restriction site of pBE-S using standard molecular cloning techniques. Next, the p43 promoter, R0, and a restriction site were inserted upstream of the peptide of interest using Gibson assembly. The p43 promoter and R0 RBS were selected because we had previously found them to be operational in GP01252 and GP01336 as well as various Bacillus spp. isolates.

The assembly was transformed into E. coli MC1061 F′ and resulting colonies were verified using colony PCR and Sanger sequencing. A transformant containing the correctly assembled plasmid was selected and cultured at 37° C. overnight. The final assembled plasmid was then isolated using a standard miniprep protocol.

The plasmid miniprep was digested using MluI and EagI which removed a fragment directly upstream of the mature antimicrobial peptide gene which would serve as the insertion location for the library of Bacillus secretion tags. The digest was run on a 0.8% agarose gel, excised, then gel purified.

The digested plasmid was then placed in a ligation reaction with the secretion tag mix (Takara Bio) using the In-Fusion HD Enzyme Mix (Takara Bio). The reaction used a 2:1 insert to vector ratio and was incubated at 50° C. for 15 minutes. After incubation, the ligation product was transformed in E. coli HST08 using heat shock. Transformants were plated on LB with ampicillin (100 ug/mL) and incubated at 37° C. overnight.

The next day, the transformant colonies were resuspended with ~5 ml of LB and miniprepped. The plasmid secretion tag library was transformed into the competent GP01252 and GP01336 and then plated on BHI with kanamycin (20 ug/mL) and incubated overnight.

The next day 100-200 transformants were tested using an agar diffusion assay against an appropriate indicator strain to screen for relative peptide production. The resulting zones of inhibition from the transformants were examined and isolates from the GP01336 transformation that gave the largest zones of inhibition were sent for sequencing to find the identity of the secretion tag.

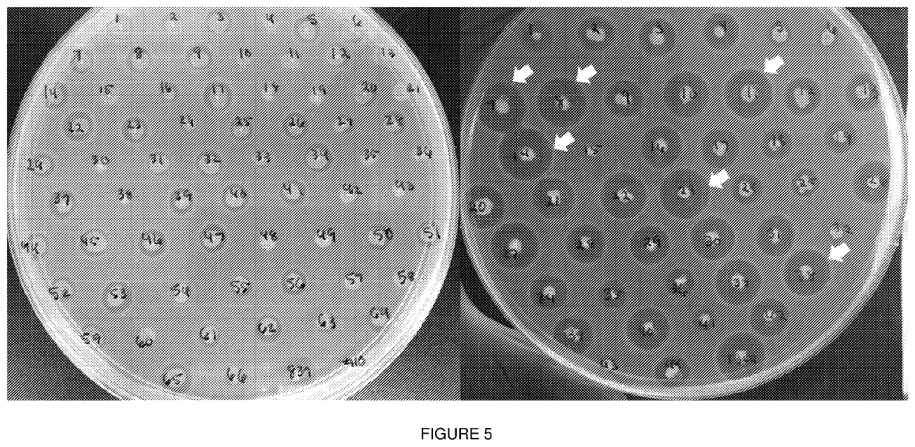

FIG. 5 shows a representative example of one of these library screens. The agar diffusion assay of the library transformants displayed a wide spectrum of inhibition ranging from no detectable inhibition to zones of inhibition ~5-10 mm in diameter. This observation demonstrates that the type of secretion tag can impact the production and secretion of the antimicrobial peptide. The white arrows indicate a group of unique secretion tags that produced the largest halos in the engineered GP01336 strain and were sequenced. Isolates 7, 8, 11, 14, 23 and 38 from the GP01336 transformation plate were discovered to use the AprE, AbnB, Mdr, EgIS, AbnA, and PhrC secretion tag to export enterocin A, respectively. Secretion tags from this set are contemplated for inclusion in the final Bacillus product derived from GP01336.

### Example 12: Introduction of Rifampicin Marker in Isolates

To assist in the enumeration of the probiotic during development, the producer strain was marked with resistance to rifampicin. This was only performed on GP01336 and not GP01252. This was done through selection of spontaneous mutants on growth agar containing the antibiotic of interest at the concentration of interest.

Briefly, the GP01336 was struck out on a BHI agar and was incubated overnight at 37° C. A fresh colony was inoculated in a 3 mL BHI culture and incubated overnight under aerobic conditions at 37° C. The next day the culture was centrifuged (16,000×g, 30 sec) and resuspended in ~100 uL of media. The bacterial resuspension was spread on BHI+rifampicin (150 ug/mL) and incubated overnight at 37° C.

The following day, colonies were selected and tested for growth on BHI agar plates. Colonies exhibiting wild-type growth and morphology were then saved for long-term storage as −80° C. freezer stocks. The rifampicin-resistant version of GP01336 is designated GP01403.

### Example 13: Construction of ThyA and DapA Knockouts

Generally, plasmids are inherently unstable and are lost from the engineered strain in the absence of pressure. Typically, plasmids contain an antibiotic resistance gene and are maintained in the engineered bacteria by applying the designated antibiotic to the growth medium. Thus only cells that have maintained the plasmid are able to propagate.

The use of a dedicated antibiotic resistance gene is unacceptable for bacteria that will be used in human or animal health because of the risk of gene transfer. Additionally, the required application of an antibiotic during production may lead to increased production costs and risk of antibiotic contamination in the final product.

An alternative method for plasmid maintenance was implemented for selected Bacillus isolates. In this method, an essential gene was removed from the chromosome and was placed on the plasmid of interest. Examples of essential genes used for this purpose include but are not limited to dapA (4-hydroxy-tetrahydrodipicolinate synthase), thyA (thymidylate synthase A), or homologues thereof. DapA and thyA are involved in diaminopimelic acid and thymidine synthesis respectively. This system was implemented for GP01403. The construction the new plasmids and the chromosomal modifications of the isolates is detailed below.

A plasmid was assembled to contain the pKS1 backbone, a spectinomycin resistance gene, and flanking regions targeting the gene of interest in GP01403. The pKS1 backbone contains a temperature sensitive origin of replication compatible with both E. coli and Bacillus. The plasmid was assembled and transformed in MC1061 F′. The strain was grown at 30° C. under aerobic conditions in BHI+spectinomycin (100 ug/mL). Note that cells were grown at 30° C. rather than 37° C. to enable plasmid replication.

After confirmation of proper assembly via colony PCR, the plasmid was miniprepped and transformed into GP01403 (see Example 9 for protocol). After confirmation of the presence of the plasmid in the given strain, a colony was selected and grown in BHI+spectinomycin at 37° C. shaking for 24 hours. The increase of temperature would inhibit replication of plasmid but the presence of spectinomycin would force the whole plasmid to integrate into the target gene locus. This is the first recombination step at the thyA/dapA gene locus.

After the 24 hr incubation, the culture was serially diluted, and the 10⁰-10⁴ dilutions were plated on BHI agar+spectinomycin and incubated overnight at 37° C. The next day, colonies were tested for proper plasmid integration into the chromosome using colony PCR. Colonies exhibiting proper integration were inoculated into 3 mL liquid BHI cultures and were incubated under aerobic conditions at 30° C. for 24 hr. This second incubation period allowed for the second recombination step in which the plasmid backbone was excised from the chromosome. Thus the resulting knockout strain was left with only regions flanking the gene of interest.

After the 24 hr incubation period, the culture was serially diluted, and the 10⁰-10⁴ dilutions were plated on BHI+diaminopimelic acid (50 ug/mL) for dapA knockouts or BHI+thymidine (50 ug/mL) and trimethoprim (10 ug/mL) for thyA knockouts and incubated at 37° C. overnight. The trimethoprim assists in eliminating cells that revert back to the wild-type genotype.

The next day, prospective diaminopimelic acid and thymidine auxotroph colonies were confirmed via colony PCR of the dapA or thyA gene locus. Isolate growth was also tested on BHI+diaminopimelic acid or thymidine+spectinomycin to confirm that the plasmid was excised from the chromosome and on BHI lacking diaminopimelic acid or thymidine to confirm the isolates were in fact auxotrophic for the nutrient of interest. The generation of the thymidine auxtrophs for GP01403 was ultimately successful and the thymidine auxotroph of GP01403 is designated GP01415.

### Example 14: Testing Bacillus spp. Transformants for Peptide Production

Table 8 lists recombinant live Bacillus strains tested in various activity assays, including ones described in this Example.

TABLE 8
Bacillus strains engineered and tested in antimicrobial assays

| Antimicrobioal | | | | | | |
|---|---|---|---|---|---|---|
| peptide (AMP) | Carrier | Wild-Type | Secretion | Selective | | Rifampicin |
| System | Organism | Strain | Tag | Pressure | Plasmid | Resistance |
| GP01270 | GP01252 | GP01252 | AmyQ | Kan | pKG293 | No |
| | | | | | (SEQ ID NO: 44) | |
| GP01284 | GP01252 | GP01252 | LipA | Kan | pSM504 | No |
| GP01324 | GP01336 | GP01336 | AmyQ | Kan | pKG293 | No |
| GP01342 | GP01336 | GP01336 | LipA | Kan | pSM504 | No |
| GP01346 | GP01336 | GP01336 | AprE | Kan | pSM547 | No |
| GP01347 | GP01336 | GP01336 | AbnB | Kan | pSM548 | No |
| GP01348 | GP01336 | GP01336 | Mdr | Kan | pSM549 | No |
| GP01349 | GP01336 | GP01336 | EgIS | Kan | pSM550 | No |
| GP01350 | GP01336 | GP01336 | AbnA | Kan | pSM551 | No |
| GP01351 | GP01336 | GP01336 | PhrC | Kan | pSM552 | No |
| GP01416 | GP01415 | GP01336 | AmyQ | ThyA (p43) | pSM576 | Yes |
| | | | | | (SEQ ID NO: 45) | |
| GP01417 | GP01415 | GP01336 | AmyQ | ThyA (pylB) | pSM583 | Yes |
| GP01418 | GP01415 | GP01336 | AmyQ | ThyA (pgsiB) | pSM584 | Yes |
| GP01420 | GP01415 | GP01336 | LipA | ThyA (p43) | pSM587 | Yes |
| GP01434 | GP01415 | GP01336 | AprE | ThyA (p43) | pSM595 | Yes |
| GP01435 | GP01415 | GP01336 | AbnB | ThyA (p43) | pSM596 | Yes |
| GP01436 | GP01415 | GP01336 | Mdr | ThyA (p43) | pSM597 | Yes |
| GP01437 | GP01415 | GP01336 | EgIS | ThyA (p43) | pSM598 | Yes |
| GP01438 | GP01415 | GP01336 | AbnA | ThyA (p43) | pSM599 | Yes |
| GP01439 | GP01415 | GP01336 | PhrC | ThyA (p43) | pSM600 | Yes |

### Agar Diffusion Assays

Transformant colonies were selected from each of the engineered systems that utilize GP01252, GP01336, or GP01415 as a chassis and were tested for antimicrobial activity against the appropriate indicator strain (ex. E. faecium 8E9 or L. monocytogenes CDC 7762. The indicator strain plates were made by inoculating molten BHI agar with a concentration of 0.3-1 uL overnight culture/mL agar depending on the indicator strain. Approximately 15-20 transformant colonies were tested from each strain and each plate included a positive control for peptide production (i.e. a strain known to produce the peptide of interest) and the unmodified GP01252 or GP01336 strain as comparison. GP01416 produced the largest halos and was, therefore, incorporated into an MIC supernatant assay (described below). An example of an agar diffusion assay depicting some of the engineered systems is shown in FIG. 4.

### Supernatant MIC Assays

Cultures of GP01336 and GP01416 were grown in BHI+25 w/v % skim milk at 37° C. overnight (~18 hours). Cultures were centrifuged at ~20,000 rpm for 20 minutes to pellet cells and milk particles. The isolated supernatants were boiled for 10 minute at 100° C. then filtered (0.22 μM PES filter) to eliminate any spores that may have remained intact from the boiling.

60 uL of the treated supernatant samples were then placed into the wells of a 96 well plate. 2× dilutions were performed on supernatants using phosphate-buffered saline. An overnight culture of indicator strain (ex. E. faecium or Listeria monocytogenes) was then diluted in fresh growth medium (BHI) to a concentration of ~1000 CFU/mL. 270 μl of diluted indicator strain was then added to each well. The plates were incubated overnight at 37° C. and the level of inhibition was analyzed the next day.

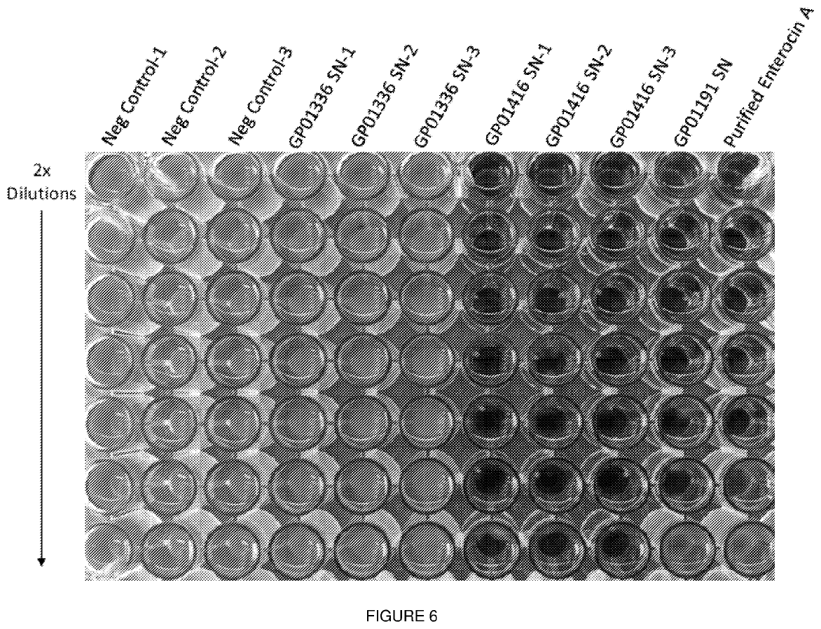

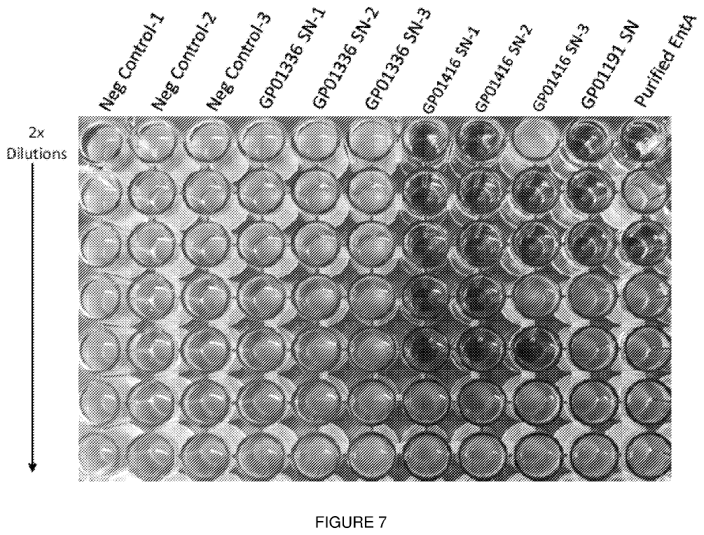

Supernatant from an enterocin A-producing E. coli strain called GP01191 was used as a positive control for activity. Additionally, purified enterocin A of a known concentration was added to one of the columns to enable absolute peptide quantification from the GP01416 supernatant. FIGS. 6 and 7 show the MIC assay results comparing GP01416 and GP01336 supernatant. against E. faecium and L. monocytogenes respectively.

### Example 15: Effectiveness of Recombinant Live Bacillus Spp. Against Clostridium perfringens

After testing the GP01416. against common enterocin A indicator strains, GP01416 was tested against various strains of Clostridium perfringens. Note that tests are performed first on indicator strains because these strains exhibit more robust growth and thus more quantitative, reproducible comparisons of peptide activity can be performed compared to C. perfringens. Additionally, GP01252 along with its engineered counterpart GP01270 were tested against C. perfringens NAH-JP1011

GP01252, GP01336, and GP01416 were struck out on BHI along with GP01270 on BHI+kanamycin (20 ug/mL). The various C. perfringens strains. were inoculated from freezer stocks into 15 ml conical tubes containing thioglycollate+10% beef extract. Conical tubes were sealed tightly and incubated statically overnight at 37° C.

The next day, 100 uL of each of the C. perfringens. cultures was spread on top of BHI agar plates and allowed to dry. After the plates were dried, five replicates of GP01252, GP01270, GP01336 and GP01416 were stabbed onto the C. perfringens. strain plates. The plates were sealed in air-tight bags with anaerobic sachets and incubated overnight at 37° C.

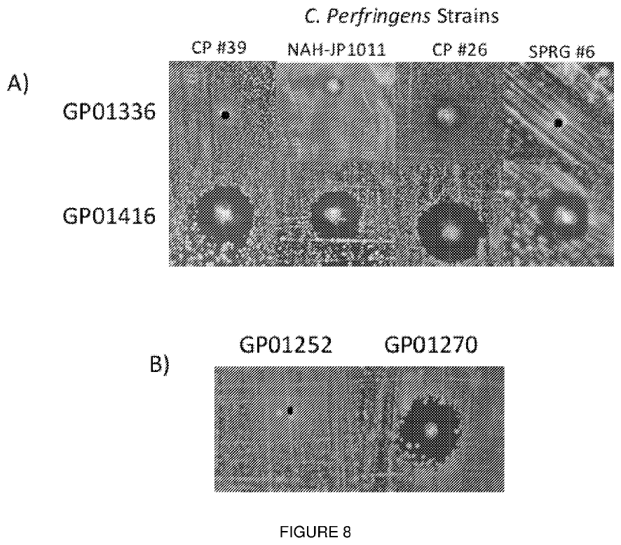

The plates and zones of inhibition for the Bacillus strains were analyzed the next day. GP01416 was considered to show improved activity against C. perfringens. GP01336 has some inhibitory effect against some C. perfringens strains but GP01416 produced consistently larger halos noticeably different from those of GP01336. GP01270 produced a good zone of inhibition against NAH-JP1011 and GP01252 produced no halo indicating no innate activity. FIG. 8 shows the results of a typical activity assay against C. perfringens.

### Example 16: Growth Curves of Recombinant Live Bacillus Spp. Isolates in Various Environments

Genetic modification of bacterial strains can often result in hindered growth. These fitness defects can be detrimental to both manufacturing and in vivo efficacy. It is therefore prudent to test the growth of recombinant strains compared to their unmodified counterparts in relevant growth conditions.

In this example, GP01336 and GP01416 were tested in rich media typically used in laboratory assays as well as media generated from the intestinal contents of chickens. This secondary media is intended to be representative of the nutrients available in the jejunum of chickens. This is the area of interest for the final application of GP01416. As such, it is crucial that the recombinant strain be metabolically active given the nutrient in that region.

For the growth assays, GP01336 and GP01416 were struck out from freezers stocks onto BHI agar plates and incubated overnight at 37° C. The next day overnight cultures were made of the strains in 3 mL of BHI and incubated overnight shaking at 37° C.

The next day, the fresh Bacillus cultures were used to inoculate cultures for the growth assay. Growth curve assays were performed in 96 well plate format and included three replicates of each growth condition. 300 uL of the medium of interest was added to the appropriate wells and was seeded with 3 uL of fresh culture of either GP01336 or GP01416 to be tested. Growth curves were monitored using a Biotek Epoch2 spectrophotometer with continuous shaking at 37° C. Optical density readings at 600 nm (OD600) readings were taken every 15 minutes for 20 hours.

The conditions tested included used either nutrient-rich media (BHI) or jejunal contents. The jejunal content media was made using filter-sterilized intestinal contents isolated from healthy birds that had been diluted 5× in phosphate-buffered saline. The contents were then further diluted 5× in M9 minimal media.

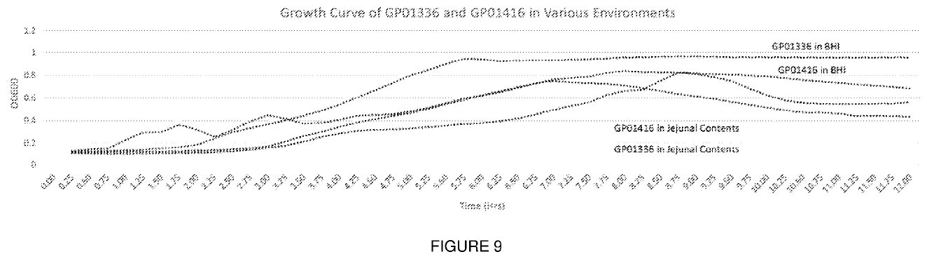

FIG. 9 shows representative growth curves of GP01336 and GP01416 in rich medium and jejeunal contents. It was observed that GP01336 reached exponential phase faster than GP01416 in both conditions. Despite differences in growth rates however, robust, dense growth was observed for both strains in both sets of nutrients.

In addition to testing different media, varying concentrations of Tween 80 were tested (0, 0.025, 0.05, and 0.075%) to decrease co-aggregation in the wells. Tween 80 concentration helped decrease the coaggregation and the cells did not seem to be strongly affected by the presence of the Tween 80 in the nutrient rich media. However, the Tween 80 seemed to negatively impact the growth of the cells in the jejunal contents.

### Example 17: Use of Recombinant Live Bacillus spp. to Treat Broilers in a Necrotic Enteritis Floor Pen Challenge Model

The recombinant live Bacillus sup. are administered to poultry to reduce C. perfringens-induced necrotic enteritis. This example outlines a typical study protocol used to demonstrate the efficacy of the recombinant Bacillus isolates in reducing necrotic enteritis and improving the and productivity parameters for poultry producers.

### Objectives/Purpose of Animal Study

### Primary

To demonstrate the efficacy of recombinant live Bacillus spp. strains on the control of gross lesion and mortality due to necrotic enteritis, when medicated through water continuously from 0 to 42 days, in broilers raised in floor pens.

### Secondary

To demonstrate the efficacy of recombinant live Bacillus spp. strains on average daily gain, final body weight and feed efficiency (FE) under necrotic enteritis challenge conditions, when medicated through water continuously from 0-42 days, in broilers raised in floor pens.

This study is conducted in 60 floor pens each with the dimension of 4′ x 4′. The study utilizes 1,200 commercial male broilers (Ross 708). Each pen is an experimental unit.

The study begins on Day 0 (arrival) and ends on Day 42.

There are three treatment groups (one negative control without challenge, one negative control with challenge, and one test article group with challenge level) as shown in Table 9.

Each treatment group uses 20 pens, i.e., replicates. Each pen houses 20 birds on day 0. Treatment groups are represented as G01, G02, and G03.

On study day 0, twenty apparently healthy chicks without any physical deformities are enrolled in each pen.

Treatment group G03 are medicated with the recombinant live Bacillus spp. test article from Day 0-41 continuously at the rate of 10⁸ CFU/bird/day.

The G02 and G03 treatment groups are challenged with C. perfringens strain at the rate of 10⁷ CFU/mL/bird on day 18 through oral gavage.

On day 42, ceca, jejunum, and ileum are collected from ten birds from each of the pens for G01, G02, and G03.

Intestinal sections are placed into separate collection bags, and fecal (cecal content) samples are collected in separate tubes.

Body weight and feed consumption data are collected on Day 14, Day 21, Day 29 and Day 42. Water consumption data are collected daily. At the end of the study, all the remaining birds are humanely euthanized and composted.

TABLE 9
Treatment Groups

| | | C. perfringens | | | Total |
|---|---|---|---|---|---|
| Treatment | | Challenge | Replicate | Birds per | Number |
| Group | Description | (Dose) | Pens | Pen | of Birds |
| G01 | Non-medicated, Non- | No (NA) | 20 | 20 | 400 |
| | challenged Control (NNC) | | | | |
| G02 | Non-medicated, Challenge | Yes (10⁷) | 20 | 20 | 400 |
| | Control (NCC) | | | | |
| G03 | Bacillus Water Additive | Yes (10⁷) | 20 | 20 | 400 |
| | (Days 0-42), once daily | | | | |

### Example 18: B. subtilis Proteases that Inhibit Bacteriocin Activity

Bacillus subtilis secretes a series of proteases that can indiscriminately degrade various proteins in the surrounding environment. B. subtilis 168 is no exception and several extracellular protease mutants have been made to improve heterologous protein secretion. Some of these B. subtilis mutants include RIK1285 (ΔnprB and ΔnprE) and WB800 (also known as Δ8) (ΔnprB, ΔnprE, ΔaprE, Δvpr, Δbpr, Δepr, Δmpr, and ΔwprA). Enterocin A is particularly vulnerable to degradation due to its small linear structure and efforts to produce sufficient antimicrobial activity using a wild-type B. subtilis chassis have been unsuccessful.

An in-depth look was taken at the extracellular proteases which were deleted in the RIK1285 and WB800 strains to determine if these proteases could play a significant role in enterocin A degradation. The MEROPS database (https://www.ebi.ac.uk/merops/) was used to examine the family origins and cleavage motifs of the eight proteases of interest. Expasy PeptideCutter (https://web.expasy.org/peptide_cutter/) was a database used to input the amino acid sequence of mature enterocin A and recover which proteases targeted the sequence along with the number of cleavage positions.

The first major protease family is called the peptidase S8 family that include neutral to mildly alkaline serine proteases. These proteases are non-specific and are thought to cleave after any hydrophobic amino acid. Five of the eight protease genes belong to this family and include aprE (family type), vpr, bpr, wprA, and epr. Due to their non-specific nature, it is most likely they play a significant role in the degradation of mature enterocin A in the extracellular environment. The next the family of proteases include the peptidase family M4 that mostly contain metallopeptidases which require a metal ion, such as zinc, for activity. NprB and nprE encode proteins belonging to this family and the preferred cleavage site is set up as Xaa+Yaa in which Xaa is a hydrophobic residue and Yaa is either a leucine, phenylalanine, isoleucine, or valine. The family type peptidase is thermolysin and is shown to have 11 target sites on mature enterocin A. The last protease family, to which the encoding protein of mpr is a member of, is the peptidase family S1 containing another family of serine proteases. The preferred cleavage site for this family is dependent on the P1 position and is divided into three categories: trypsin-like, chymotrypsin-like (high and low specificity), and elastase-like. A trypsin-like cleavage occurs if there is an arginine or lysine in the P1 position (5 sites on mature enterocin A). A chymotrypsin-like cleavage occurs if there is a tryptophan, tyrosine, or phenylalanine at the P1 position (5 sites on mature enterocin A) and to a lesser extent leucine, methionine, or histidine (8 sites on mature enterocin A). An elastase-like cleavage occurs when an alanine is found at P1 position.

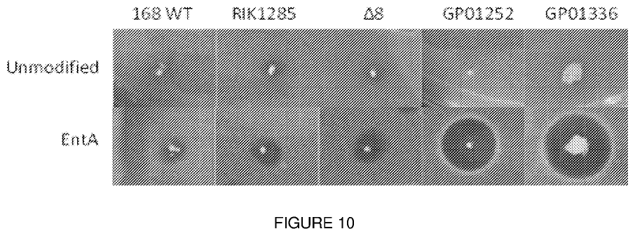

Overall, it was found that the proteases knocked out in the protease mutants belong to protease families that most likely target mature enterocin A at multiple sites. This results in a degradation rate that would clearly outpace the rate of secretion. Additionally, it appears that cleavage sites for proteases are more ambiguous with a hierarchy of preferences compared to the site requirements of restriction endonucleases. The enterocin A constructs were thus transformed into these protease-deficient mutants to decreasing enterocin A degradation and improve activity. FIG. 10 shows halo assays against the indicator strain Enterococcus faecium 8E9 using various B. subtilis 168 strains carrying the same enterocin A construct.

The halo assay showed slight improvement in halo size from the protease mutants RIK1285 and A8. This indicates that the proteases knocked out in the mutants participated in the degradation of enterocin A. However, there halo sizes of 48 were still much smaller compared to GP01336 and GP01252 (another Bacillus spp. isolate transformed with an enterocin A construct). This could indicate that there is still significant enterocin A degradation even in an eight-protease knockout.

The sequences of the eight protease genes from B. subtilis 168 were compared the genome of our Bacillus paralicheniformis isolate, GP01336, using Basic Local Alignment Search Tool (BLAST). It was discovered that GP01336 had no homologs for the nprE or nprB genes. Additionally, the blasted amino acid sequence for the Mpr protein showed a low amount of homology (~44%) to a glutamyl endopeptidase precursor found in the genome of GP01336.

Using Expasy PeptideCutter, it was found that glutamyl endopeptidase should not cleave mature enterocin A. The absence of homologs for the nprE and nprB genes coupled with the low homology found for the Mpr protein may make this strain more efficient at secreting intact mature enterocin A. In terms of the non-specific proteases, GP01336 contained homologs for AprE, WprA, Vpr, Bpr, and Epr proteins with greater protein homology (65%, 49%, 71%, 62%, and 54% respectively). There may be greater homology with this set of proteases because the strain requires them to obtain free amino acids vital for protein synthesis, however, proteases found in GP01336 may be different enough from those found in 168 to have less of an affinity towards mature enterocin A.

High temperature requirement A and B (HtrA and HtrB) are two common membrane-bound proteases found in Bacillus subtilis. MEROPS indicates that these are serine proteases belong to the peptidase S1 family. These proteases are unique in the fact that the proteins are activated in the event of secretion stress and the overproduction of α-amylase. These proteases can present an issue in the biotechnology industry because the overproduction of heterologous proteins can cause burden on the cell. In the case of the construct herein, the overproduction of enterocin A may activate the expression of HtrA and HtrB leading to degradation. A comparison of the HtrA and HtrB genes from B. subtilis to homologs found in GP01336 shows a protein homology of 62 and 63% respectively. It is contemplated herein that the homologs found in GP01336 have different requirements for activation that leave mature enterocin A relatively intact.

Amino acid sequences are shown herein for Bacillus proteases NprB (SEQ ID NO: 34), NprE (SEQ ID NO: 35), WprA (SEQ ID NO: 36), Bpr (SEQ ID NO: 37), AprE (SEQ ID NO: 38), Epr (SEQ ID NO: 39), Mpr (SEQ ID NO: 40), Vpr (SEQ ID NO: 41), HtrA (SEQ ID NO: 42), and HtrB (SEQ ID NO: 43).

We claim:

1. A composition for treating a Clostridia perfringens-induced disease condition comprising a Bacillus bacterium isolated from an intestinal tract of an animal and transformed with an exogenous polynucleotide, wherein the exogenous polynucleotide comprises a heterologous promoter operably linked to a polynucleotide that encodes a secretion tag fused to an antimicrobial peptide with bacteriolytic or bacteriostatic activity against Clostridia perfringens.

2. The composition of claim 1, wherein the bacterium is a Bacillus spp. bacterium.

3. The composition of claim 2 wherein the Bacillus bacterium is a Bacillus oleronius strain GP01252 bacterium transformed with the exogenous polynucleotide.

4. The composition of claim 2, wherein the Bacillus bacterium is Bacillus licheniformis, Bacillus paralicheniformis. Bacillus oagulans, Bacillus pumilus, Bacillus clausii, or Bacillus anyliquefaciens transformed with the exogenous polynucleotide.

5. The composition of claim 4 wherein the Bacillus paralicheniformis bacterium is a Bacillus paralicheniformis strain GP01336 bacterium (ATCC Accession No. PTA-127307) transformed with the exogenous polynucleotide

6. The composition of any preceding claim, wherein the Bacillus bacterium does not contain the genes of proteases NprE, NprB, AprE, WprA, Vpr, Bpr, Epr, HtrA, and HtrB with greater than 70%, greater than 60%, or greater than 50% homology to these genes present in Bacillus subtilis 168.

7. A composition of any preceding claim, wherein the antimicrobial peptide is a Class II bacteriocin.

8. The composition of any preceding claim, wherein the Clostridia perfringens-induced disease is necrotic enteritis.

9. A composition of any of claim 1-6 or 8, wherein the antimicrobial peptide is a class I bacteriocin.

10. A composition of any of claims 1-8, wherein the antimicrobial peptide is enterocin A, enterocin B, enterocin P, carnobactreiocin B, plantaricin EF, or hiracin JM79.

11. The composition of any preceding claim, wherein the heterologous promoter is the constitutive promoter p43.

12. The composition of any preceding claim, wherein the exogenous polynucleotide that encodes the antimicrobial protein is a plasmid.

13. The composition of claim 12, wherein the Bacillus bacterium is a thymidine auxotroph and the selective pressure on the plasmid in the bacterium is a thymidylate synthase A (ThyA) gene under the control of a heterologous promoter.

14. The composition of claim 13, wherein the heterologous promoter is either the p43, pylB, or pgsiB promoter.

15. The composition of any of claims 1-12, wherein the Bacillus bacterium is a prototroph and the selective pressure on the plasmid in the bacterium is a kanamycin resistance gene.

16. The composition of any preceding claim further comprising a pharmaceutically acceptable carrier.

17. A method for treating a disease condition caused by Clostridia perfringens in an animal, comprising administering to the animal in need thereof, the composition of any of claims 1-16.

18. The method of claim 17 wherein the disease is necrotic enteritis.

19. The method of claim 17, wherein the animal is a mammal.

20. The method of claim 17, wherein the mammal is a human, dog, cat, or pig.

21. The method of claim 17, wherein the animal is a bird.

22. The method of claim 17, wherein the bird is a chicken, turkey or duck.

23. The method of claim 17, wherein the animal is a fish.

24. A method for restoring rate of weight gain in an animal that has necrotic enteritis caused by Clostridia perfringens, comprising the step of administering to an animal in need thereof, the composition of any of claims 1-16.

25. The method of claim 24, wherein the animal is a mammal.

26. The method of claim 24, wherein the mammal is a pig.

27. The method of claim 24, wherein the animal is a bird.

28. The method of claim 24, wherein the bird is a chicken, turkey or duck.


SEQUENCE LISTING (53 sequences)

SEQ ID NO: 1 - protein, 47 aa, synthetic construct
  REGION 1..47: note = Synthetic
TTHSGKYYGNGVYCTKNKCTVDWAKATTCIAGMSIGGFLGGAIPGKC

SEQ ID NO: 2 - protein, 44 aa, synthetic construct
  REGION 1..44: note = Synthetic
ATRSYGNGVYCNNSKCWVNWGEAKENIAGIVISGWASGLAGMGH

SEQ ID NO: 3 - protein, 53 aa, synthetic construct
  REGION 1..53: note = Synthetic
ENDHRMPNELNRPNNLSKGGAKCGAAIAGGLFGIPKGPLAWAAGLANVYSKCN

SEQ ID NO: 4 - protein, 44 aa, synthetic construct
  REGION 1..44: note = Synthetic
ATYYGNGLYCNKEKCWVDWNQAKGEIGKIIVNGWVNHGPWAPRR

SEQ ID NO: 5 - protein, 53 aa, synthetic construct
  REGION 1..53: note = Synthetic
DQMSDGVNYGKGSSLSKGGAKCGLGIVGGLATIPSGPLGWLAGAAGVINSCMK

SEQ ID NO: 6 - protein, 48 aa, synthetic construct
  REGION 1..48: note = Synthetic
VNYGNGVSCSKTKCSVNWGQAFQERYTAGINSFVSGVASGAGSIGRRP

SEQ ID NO: 7 - protein, 46 aa, synthetic construct
  REGION 1..46: note = Synthetic
AAPKITQKQKNCVNGQLGGMLAGALGGPGGVVLGGIGGAIAGGCFN

SEQ ID NO: 8 - protein, 43 aa, synthetic construct
  REGION 1..43: note = Synthetic
IGGALGNALNGLGTWANMMNGGGFVNQWQVYANKGKINQYRPY

SEQ ID NO: 9 - protein, 33 aa, synthetic construct
  REGION 1..33: note = Synthetic
FNRGGYNFGKSVRHVVDAIGSVAGIRGILKSIR

SEQ ID NO: 10 - protein, 34 aa, synthetic construct
  REGION 1..34: note = Synthetic
VFHAYSARGVRNNYKSAVGPADWVISAVRGFIHG

SEQ ID NO: 11 - protein, 25 aa, synthetic construct
  REGION 1..25: note = Synthetic
GAWKNFWSSLRKGFYDGEAGRAIRR

SEQ ID NO: 12 - protein, 32 aa, synthetic construct
  REGION 1..32: note = Synthetic
RRSRKNGIGYAIGYAFGAVERAVLGGSRDYNK

SEQ ID NO: 13 - protein, 31 aa, synthetic construct
  REGION 1..31: note = Synthetic
MIQKRKRTVSFRLVLMCTLLFVSLPITKTSA

SEQ ID NO: 14 - protein, 32 aa, synthetic construct
  REGION 1..32: note = Synthetic
MKFVKRRIIALVTILMLSVTSLFALQPSAKAA

SEQ ID NO: 15 - protein, 35 aa, synthetic construct
  REGION 1..35: note = Synthetic
MDTTTAKQASTKFVVLGLLLGILMSAMDNTIVATA

SEQ ID NO: 16 - protein, 29 aa, synthetic construct
  REGION 1..29: note = Synthetic
MRSKKLWISLLFALTLIFTMAFSNMSAQA

SEQ ID NO: 17 - protein, 27 aa, synthetic construct
  REGION 1..27: note = Synthetic
MKLKSKLFVICLAAAAIFTAAGVSANA

SEQ ID NO: 18 - protein, 32 aa, synthetic construct
  REGION 1..32: note = Synthetic
MKKKKTWKRFLHFSSAALAAGLIFTSAAPAEA

SEQ ID NO: 19 - protein, 26 aa, synthetic construct
  REGION 1..26: note = Synthetic
MFNRLFRVCFLAALIMAFTLPNSVYA

SEQ ID NO: 20 - protein, 29 aa, synthetic construct
  REGION 1..29: note = Synthetic
MKRSISIFITCLLITLLTMGGMIASPASA

SEQ ID NO: 21 - protein, 51 aa, synthetic construct
  REGION 1..51: note = Synthetic
MAYDSRFDEWVQKLKEESFQNNTFDRRKFIQGAGKIAGLSLGLTIAQSASA

SEQ ID NO: 22 - protein, 44 aa, synthetic construct
  REGION 1..44: note = Synthetic
MSDEQKKPEQIHRRDILKWGAMAGAAVAIGASGLGGLAPLVASA

SEQ ID NO: 23 - DNA, 96 bp, synthetic construct
  misc_feature 1..96: note = Synthetic
  source 1..96: mol_type = other DNA
aattttgtcaaaataattttattgacaacgtcttattaacgttgatataatttaaattttatttgacaaaaatgggctcgtgttgtacaataaatg

SEQ ID NO: 24 - DNA, 258 bp, synthetic construct
  misc_feature 1..258: note = Synthetic
  source 1..258: mol_type = other DNA
tgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatata

SEQ ID NO: 25 - DNA, 280 bp, synthetic construct
  misc_feature 1..280: note = Synthetic
  source 1..280: mol_type = other DNA
ctatcgagacacgtttggctggaaaaaacttttccagatagtgccggttgccggaatggtttttggcgccgctgccaatcgctcaacattaaacgacattaccgagacaggcatgatgctgtacaaaaagaggcgcattcttgaacgactgaaagaaacagaacgagagatggaatagcagaaagcagacggacaccgcgatccgcctgctttttttagtggaaacatacccaatgtgttttgtttgtttaaaagaattgtgagcgggaatacaacaacc

SEQ ID NO: 26 - DNA, 152 bp, synthetic construct
  misc_feature 1..152: note = Synthetic
  source 1..152: mol_type = other DNA
ccgataattccaattttcatgatgtcacacccaatttagcatttacgtattatcatagcagaagtaagaagaaattacttctcaaagatcccatgtgcttaaaattaaagtttaaatatttggattttttaaataaagcgtttacaatatat

SEQ ID NO: 27 - DNA, 1545 bp, synthetic construct
  misc_feature 1..1545: note = Synthetic
  source 1..1545: mol_type = other DNA
aattggccgactttagatattttcgttataaagaaaaataatttgcacatgaaaaaggagatttctattttagaactcctttttcatatgagaaggtgccatgtcactattgcttcagaaatactcctagaataaaaaaactcatctttaaagatgagctgtccattccataaaaaattacattgtaatcatgtccagaaaatgatcaatcacaatggaggacattcctaatgccggtgcattctgtcctaaggaagatggcaataattcatagctattgcctaattgggaataaacccttgatgatacttcacttctcattgaatttaaaaccataggatgcgattcaattatgctatttcttaaaattacggcttgtgggttgaaagtatttagaatattggtaaggcctattcctaaatagaatccaaaattttgtaatgcatttaaggttccgatatcattcagatgggcgaggtttatgatatcttgataggacagttttttctctttggtctgaagagattttaataaagccttctctgaagcatacaattcccagcatcctcggtttccgcaactgcatttaggaccattaaagtctattgtcatatgtcccatttctccagagaagccgcttactcctctatataaatgattgttgataataacaccgatccctattcctgtgctgatacttacgtaaataatgttatcgtgattttttgcagctccaaatactttttctccatatgcgccagcatttgcctcattttcaataaaaacaggcacattgtacttctcttgtatcgaagattttaagtcaatatctctccagttggagttcggagtgaaaacaattttttgatctttatcaatgagtccaggcacgcaaatacctataccaataagcccgtacggagattggggcatttgcgtaataaagtgatgaatcatatcaatcaaaatgtctttcgttatttctggagaattggattccaaatggcggtattgatcaagaacgattgttccttcaaggtctgttaaaatgccattaatataatccacaccaacatctattccaacggagtatcctgcctttttattaaaaacaagcatgacaggtcttcttccgccacttgattgtccttgacctatttcaaataccatactttctttcattaacgtgtttacctgtgatgagacagttgatttatctaatccagtcatttcagataattttgctcttgaaataggtgaatttttaaggatttcttttaataataacttttgatttacttttttgacaaaggtttgatcagcgatatccacttcatccactccatttgtttaatctttaaattaagtatcaacatagtacatagcgaatcttccctttattatatctaatgtgttcataaaaaactaaaaaaaatattgaaaatactgacgaggttatataagatgaaaataagttagtttgtttaaacaacaaactaataggtgatgtacttactatatgaaataaaatgcatctgtagaatt

SEQ ID NO: 28 - DNA, 209 bp, synthetic construct
  misc_feature 1..209: note = Synthetic
  source 1..209: mol_type = other DNA
attggccaaagcagaaaggtccgacctaattaaagaaagggaagcaagtgttcatctgtaaagggttttaaaacgccatgcctcgtgcatggcgtttttttgtgccaatgggtccggtgcgagatacgactccggtcttatataaaaatcaatctctgattcgttttgcatatcttccaacttgtataagatgaagacaaggaaaacga

SEQ ID NO: 29 - protein, 7 aa, synthetic construct
  REGION 1..7: note = Synthetic
  SITE 6: note = misc_feature - Xaa can be any naturally occurring amino acid
YGNCVXC

SEQ ID NO: 30 - protein, 7 aa, synthetic construct
  REGION 1..7: note = Synthetic
  SITE 5: note = MISC_FEATURE - Xaa is Val or Lys
  SITE 6: note = misc_feature - Xaa can be any naturally occurring amino acid
YGNCXXC

SEQ ID NO: 31 - DNA, 22 bp, synthetic construct
  misc_feature 1..22: note = Synthetic
  source 1..22: mol_type = other DNA
ctacggcaaggcgacgctgacg

SEQ ID NO: 32 - DNA, 20 bp, synthetic construct
  misc_feature 1..20: note = Synthetic
  source 1..20: mol_type = other DNA
agagtttgatcmtggctcag

SEQ ID NO: 33 - DNA, 19 bp, synthetic construct
  misc_feature 1..19: note = Synthetic
  source 1..19: mol_type = other DNA
ggytaccttgttacgactt

SEQ ID NO: 34 - protein, 538 aa, synthetic construct
  REGION 1..538: note = Synthetic
MRNLTKTSLLLAGLCTAAQMVFVTHASAEESIEYDHTYQTPSYIIEKSPQKPVQNTTQKESLFSYLDKHQTQFKLKGNANSHFRVSKTIKDPKTKQTFFKLTEVYKGIPIYGFEQAVAMKENKQVKSFFGKVHPQIKDVSVTPSISEKKAIHTARRELEASIGKIEYLDGEPKGELYIYPHDGEYDLAYLVRLSTSEPEPGYWHYFIDAKNGKVIESFNAIHEAAGTGIGVSGDEKSFDVTEQNGRFYLADETRGKGINTFDAKNLNETLFTLLSQLIGYTGKEIVSGTSVFNEPAAVDAHANAQAVYDYYSKTFGRDSFDQNGARITSTVHVGKQWNNAAWNGVQMVYGDGDGSKFKPLSGSLDIVAHEITHAVTQYSAGLLYQGEPGALNESISDIMGAMADRDDWEIGEDVYTPGIAGDSLRSLEDPSKQGNPDHYSNRYTGTEDYGGVHINSSIHNKAAYLLAEGGVHHGVQVEGIGREASEQIYYRALTYYVTASTDFSMMKQAAIEAANDLYGEGSKQSASVEKAYEAVGIL

SEQ ID NO: 35 - protein, 521 aa, synthetic construct
  REGION 1..521: note = Synthetic
MGLGKKLSVAVAASFMSLSISLPGVQAAEGHQLKENQTNFLSKNAIAQSELSAPNDKAVKQFLKKNSNIFKGDPSKRLKLVESTTDALGYKHFRYAPVVNGVPIKDSQVIVHVDKSDNVYAVNGELHNQSAAKTDNSQKVSSEKALALAFKAIGKSPDAVSNGAAKNSNKAELKAIETKDGSYRLAYDVTIRYVEPEPANWEVLVDAETGSILKQQNKVEHAAATGSGTTLKGATVPLNISYEGGKYVLRDLSKPTGTQIITYDLQNRQSRLPGTLVSSTTKTFTSSSQRAAVDAHYNLGKVYDYFYSNFKRNSYDNKGSKIVSSVHYGTQYNNAAWTGDQMIYGDGDGSFFSPLSGSLDVTAHEMTHGVTQETANLIYENQPGALNESFSDVFGYFNDTEDWDIGEDITVSQPALRSLSNPTKYNQPDNYANYRNLPNTDEGDYGGVHTNSGIPNKAAYNTITKLGVSKSQQIYYRALTTYLTPSSTFKDAKAALIQSARDLYGSTDAAKVEAAWNAVGL

SEQ ID NO: 36 - protein, 894 aa, synthetic construct
  REGION 1..894: note = Synthetic
MKRRKFSSVVAAVLIFALIFSLFSPGTKAAAAGAIDQAAALENGKEQTGAMKEPEQVKWYKVTPGATDIQKNSHMALTVKSDSVLNVSVYPSKEKALKDETFEMYRSFTAEDGKSEVIFPYAWSGPYYVKVEYLGEEEPEDGGTAEAAAEAKYTIGYKGTKKQPSDLEEEEACPVEMSVDQKKSGKGILDKLRSIRDEQLSQTAEGKELTSLYYKAAPFIVAKLALNKTARNEIYQDLVTLKPLFDDVSENGASSSYKVTEKDQKAINRLYDKALQSVPSFLKEEIKKQADRLNMKQLQGKTAGAILTENNIAAKSEVQTTKVIFKVKDNKSLSSVHNEMKGFSASAQSKKDISNVKKAKKLFDNLYSFELPKDEKQNGAYTASAKRVKSAAATLSKMSNVEFAEPVQEYKSLANDIQYPYQWPLKNNGENGGVKNADVKYEPANTLLSKRKLNDTLIAVVDTGVDSTLADLKGKVRTDLGHNFVGRNNNAMDDQGHGTHVAGIIAAQSDNGYSMTGLNAKAKIIPVKVLDSAGSGDTEQIALGIKYAADKGAKVINLSLGGGYSRVLEFALKYAADKNVLIAAASGNDGENALSYPASSKYVMSVGATNRMDMTADFSNYGKGLDISAPGSDIPSLVPNGNVTYMSGTSMATPYAAAAAGLLFAQNPKLKRTEVEDMLKKTADDISFESVDGGEEELYDDYGDPIEIPKTPGVDWHSGYGRLNVMKAVSAADLQLKVNKLESTQTAVRGSAKEGTLIEVMNGKKKLGSAKAGKDNAFKVNIATQKQDQVLYLKATKGDAKTSYKVVVVKGKPSGTPKVNAVKTKDTAVKGKANSKAMIRVKNKSKKVIASAKADAKGTFSVKIKKQKAGTVLYVTAVDTDKKESKEAKVVVEK

SEQ ID NO: 37 - protein, 1433 aa, synthetic construct
  REGION 1..1433: note = Synthetic
MRKKTKNRLISSVLSTVVISSLLFPGAAGASSKVTSPSVKKELQSAESIQNKISSSLKKSFKKKEKTTFLIKFKDLANPEKAAKAAVKKAKSKKLSAAKTEYQKRSAVVSSLKVTADESQQDVLKYLNTQKDKGNADQIHSYYVVNGIAVHASKEVMEKVVQFPEVEKVLPNEKRQLFKSSSPFNMKKAQKAIKATDGVEWNVDQIDAPKAWALGYDGTGTVVASIDTGVEWNHPALKEKYRGYNPENPNEPENEMNWYDAVAGEASPYDDLAHGTHVTGTMVGSEPDGTNQIGVAPGAKWIAVKAFSEDGGTDADILEAGEWVLAPKDAEGNPHPEMAPDVVNNSWGGGSGLDEWYRDMVNAWRAADIFPEFSAGNTDLFIPGGPGSIANPANYPESFATGATDINKKLADFSLQGPSPYDEIKPEISAPGVNIRSSVPGQTYEDGWDGTSMAGPHVSAVAALLKQANASLSVDEMEDILTSTAEPLTDSTFPDSPNNGYGHGLVNAFDAVSAVTDGLGKAEGQVSVEGDDQEPPVYQHEKVTEAYEGGSLPLTLTAEDNVSVTSVKLSYKLDQGEWTEITAKRISGDHLKGTYQAEIPDIKGTKLSYKWMIHDFGGHVVSSDVYDVTVKPSITAGYKQDFETAPGGWVASGTNNNWEWGVPSTGPNTAASGEKVYGTNLTGNYANSANMNLVMPPIKAPDSGSLFLQFKSWHNLEDDFDYGYVFVLPEGEKNWEQAGVYNGKTSSWTDEEIDLSAYKGQNIQVMFNLQSDESIAKEGWYIDDVVLSDKSAGKTVKKNKLGVEKPSGKQKKKPVNPKKAKPSANTAVKHQNKAIQPQVLPLKAQVSVVETGKSTYSDQSTGQYTLKHKAGDYTLMAEAYGYQSKTQKVSLKTDQTTQANFTLEEMKKGTLKGTVINKTTGEPVTGASVYVVEDAAVEPAMTNDKGEYMLEAYEGAYTIKVAAPGYYSDEFSVELKGDVTKETALKPFVGYPGEIAYDDGTAENANSYFAAGNGWAVKMTLADGKDKGMLTGGLFRFWDTEFPDPGGTEFKVEVYDATGKDGAPGKKIAGPFNAEALRNGEWTKVDLSSKGIMVDKDFYLVYIQSKPDPYSPGLAMDETGQNSGRNWQYIDGKWQPGDKADGNYMIRALVDYEAAVPEITSPTDKSYTNKDSVTVKGNASPGTTVHIYNGEKEAGETKAAADGTFHAGIILNKGENELTATASTDNGTTDASSPITVTLDQEKPELTLDNPKDGGKTNKETLTVKGAVSDDNLKDVKVNGKKATVADGSYSARILLENGRNEIKVIATDLAGNKTTKKTVIDVNFDKPVISGLIPGEDKNLKAGESVKIAFSSAEDLDATFTIRMPLTNARASVQNATELPLREISPGRYEGYWTATSSIKAKGAKVEVIVRDDYGNETRKTANGKLNMNTEN

SEQ ID NO: 38 - protein, 381 aa, synthetic construct
  REGION 1..381: note = Synthetic
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 39 - protein, 645 aa, synthetic construct
  REGION 1..645: note = Synthetic
MKNMSCKLVVSVTLFFSFLTIGPLAHAQNSSEKEVIVVYKNKAGKETILDSDADVEQQYKHLPAVAVTADQETVKELKQDPDILYVENNVSFTAADSTDFKVLSDGTDTSDNFEQWNLEPIQVKQAWKAGLTGKNIKIAVIDSGISPHDDLSIAGGYSAVSYTSSYKDDNGHGTHVAGIIGAKHNGYGIDGIAPEAQIYAVKALDQNGSGDLQSLLQGIDWSIANRMDIVNMSLGTTSDSKILHDAVNKAYEQGVLLVAASGNDGNGKPVNYPAAYSSVVAVSATNEKNQLASFSTTGDEVEFSAPGTNITSTYLNQYYATGSGTSQATPHAAAMFALLKQRDPAETNVQLREEMRKNIVDLGTAGRDQQFGYGLIQYKAQATDSAYAAAEQAVKKAEQTKAQIDINKARELISQLPNSDAKTALHKRLDKVQSYRNVKDAKDKVAKAEKYKTQQTVDTAQTAINKLPNGTDKKNLQKRLDQVKRYIASKQAKDKVAKAEKSKKKTDVDSAQSAIGKLPASSEKTSLQKRLNKVKSTNLKTAQQSVSAAEKKSTDANAAKAQSAVNQLQAGKDKTALQKRLDKVKKKVAAAEAKKVETAKAKVKKAEKDKTKKSKTSAQSAVNQLKASNEKTKLQKRLNAVKPKK

SEQ ID NO: 40 - protein, 313 aa, synthetic construct
  REGION 1..313: note = Synthetic
MKLVPRFRKQWFAYLTVLCLALAAAVSFGVPAKAAENPQTSVSNTGKEADATKNQTSKADQVSAPYEGTGKTSKSLYGGQTELEKNIQTLQPSSIIGTDERTRISSTTSFPYRATVQLSIKYPNTSSTYGCTGFLVNPNTVVTAGHCVYSQDHGWASTITAAPGRNGSSYPYGTYSGTMFYSVKGWTESKDTNYDYGAIKLNGSPGNTVGWYGYRTTNSSSPVGLSSSVTGFPCDKTFGTMWSDTKPIRSAETYKLTYTTDTYGCQSGSPVYRNYSDTGQTAIAIHTNGGSSYNLGTRVTNDVFNNIQYWANQ

SEQ ID NO: 41 - protein, 806 aa, synthetic construct
  REGION 1..806: note = Synthetic
MKKGIIRFLLVSFVLFFALSTGITGVQAAPASSKTSADLEKAEVFGDIDMTTSKKTTVIVELKEKSLAEAKEAGESQSKSKLKTARTKAKNKAIKAVKNGKVNREYEQVFSGFSMKLPANEIPKLLAVKDVKAVYPNVTYKTDNMKDKDVTISEDAVSPQMDDSAPYIGANDAWDLGYTGKGIKVAIIDTGVEYNHPDLKKNFGQYKGYDFVDNDYDPKETPTGDPRGEATDHGTHVAGTVAANGTIKGVAPDATLLAYRVLGPGGSGTTENVIAGVERAVQDGADVMNLSLGNSLNNPDWATSTALDWAMSEGVVAVTSNGNSGPNGWTVGSPGTSREAISVGATQLPLNEYAVTFGSYSSAKVMGYNKEDDVKALNNKEVELVEAGIGEAKDFEGKDLTGKVAVVKRGSIAFVDKADNAKKAGAIGMVVYNNLSGEIEANVPGMSVPTIKLSLEDGEKLVSALKAGETKTTFKLTVSKALGEQVADFSSRGPVMDTWMIKPDISAPGVNIVSTIPTHDPDHPYGYGSKQGTSMASPHIAGAVAVIKQAKPKWSVEQIKAAIMNTAVTLKDSDGEVYPHNAQGAGSARIMNAIKADSLVSPGSYSYGTFLKENGNETKNETFTIENQSSIRKSYTLEYSFNGSGISTSGTSRVVIPAHQTGKATAKVKVNTKKTKAGTYEGTVIVREGGKTVAKVPTLLIVKEPDYPRVTSVSVSEGSVQGTYQIETYLPAGAEELAFLVYDSNLDFAGQAGIYKNQDKGYQYFDWDGTINGGTKLPAGEYYLLAYAANKGKSSQVLTEEPFTVE

SEQ ID NO: 42 - protein, 449 aa, synthetic construct
  REGION 1..449: note = Synthetic
MDNYRDENRTKGNENEVFLTKENDQSASYSARNVIHDQEKKKRGFGWFRPLLGGVIGGSLALGIYTFTPLGDHDSQDTAKQSSSQQQTQSVTATSTSSESKKSSSSSSAFKSEDSSKISDMVEDLSPAIVGITNLQAQSNSSLFGSSSSDSSEDTESGSGSGVIFKKENGKAYIITNNHVVEGASSLKVSLYDGTEVTAKLVGSDSLTDLAVLQISDDHVTKVANFGDSSDLRTGETVIAIGDPLGKDLSRTVTQGIVSGVDRTVSMSTSAGETSINVIQTDAAINPGNSGGPLLNTDGKIVGINSMKISEDDVEGIGFAIPSNDVKPIAEELLSKGQIERPYIGVSMLDLEQVPQNYQEGTLGLFGSQLNKGVYIREVASGSPAEKAGLKAEDIIIGLKGKEIDTGSELRNILYKDAKIGDTVEVKILRNGKEMTKKIKLDQKEEKTS

SEQ ID NO: 43 - protein, 458 aa, synthetic construct
  REGION 1..458: note = Synthetic
MDYRRDGQNDQHQTEPSHTEQQNTENQKLIGHSEQELLDAPVSYEAGRQETASALEMEKQETAVKKEKKRRAAWLSPILGGIIGGGLMLGIAPYLPSDQNQATETASANKQVQSDNFTTAPITNASNIADMVEDLEPTIVGISNIQTSQNNTFGTGGGSSSESESGTGSGVIFKKDSDKAYIITNNHVVEGANKLTVTLYNGETETAKLVGSDTITDLAVLEISGKNVKKVASFGDSSQLRTGEKVIAIGNPLGQQFSGTVTQGIISGLNRTIDVDTTQGTVEMNVLQTDAAINPGNSGGPLINASGQVIGINSLKVSESGVESLGFAIPSNDVEPIVDQLLQNGKVDRPFLGVQMIDMSQVPETYQENTLGLFGDQLGKGVYVKEVQANSPAEKAGIKSEDVIVKLNGKDVESSADIRQILYKDLKVGDKTTIQVLRKGKTKTLNATLTKQTESSSS

SEQ ID NO: 44 - DNA, 6274 bp, synthetic construct
  misc_feature 1..6274: note = Synthetic
  misc_feature 529..786: note = P43 Promoter
  misc_feature 790..813: note = R0 RBS
  misc_feature 815..908: note = AmyQ Sec Tag
  misc_feature 908..1051: note = Mature Enterocrin A
  misc_feature 1056..1184: note = B0015 Terminator
  source 1..6274: mol_type = other DNA
gcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttcctaagaattccacctgctgacggagtgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatatatactgattaactaataaggaggacaaaaatgattcaaaaacgaaagcggacagtttcgttcagacttgtgcttatgtgcacgctgttatttgtcagtttgccgattacaaaaacatcagccaccactcatagtggaaaatattatggaaatggagtgtattgcactaaaaataaatgtacggtcgattgggccaaggcaactacttgtattgcaggaatgtctataggtggttttttaggtggagcaattccagggaagtgctaagcttccaggcatcaaataaaacgaaaggctcagtcgaaagactgggcctttcgttttatctgttgtttgtcggtgaacgctctctactagagtcacactggctcaccttcgggtgggcctttctgcgtttatacgctcagacgctcagatgacgcaggtgctgcagtaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctatttcactttttgcattctacaaactgcataactattatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagacgtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagccttcatcacttgcctcccatagatgaatccgaacctcattacacattagaactgcgaatccatcttcatggtgaaccaaagtgaaacctagtttatcgcaataaaaacctatactctttttaatatccccgactggcaatgccgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacagtaaccaacatgattaacaattattagaggtcatcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgaagcgctcgtcgtataacagatgcgatgatgcagaccaatcaacatggcacccgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggac

SEQ ID NO: 45 - DNA, 6743 bp, synthetic construct
  misc_feature 1..6743: note = Synthetic
  misc_feature 529..786: note = P43 Promoter
  misc_feature 791..813: note = R0 RBS
  misc_feature 815..907: note = Amy Q Sec Tag
  misc_feature 907..1051: note = Mature Enterocrin A
  misc_feature 1056..1184: note = B0015 Terminator
  misc_feature 4414..4671: note = P43 Promoter
  misc_feature 4676..4698: note = R0 RBS
  misc_feature 4700..5602: note = Thymidylate Synthase A
  misc_feature 5607..5745: note = B0015 Terminator
  source 1..6743: mol_type = other DNA
gcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttcctaagaattccacctgctgacggagtgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatatatactgattaactaataaggaggacaaaaatgattcaaaaacgaaagcggacagtttcgttcagacttgtgcttatgtgcacgctgttatttgtcagtttgccgattacaaaaacatcagccaccactcatagtggaaaatattatggaaatggagtgtattgcactaaaaataaatgtacggtcgattgggccaaggcaactacttgtattgcaggaatgtctataggtggttttttaggtggagcaattccagggaagtgctaagcttccaggcatcaaataaaacgaaaggctcagtcgaaagactgggcctttcgttttatctgttgtttgtcggtgaacgctctctactagagtcacactggctcaccttcgggtgggcctttctgcgtttatacgctcagacgctcagatgacgcaggtgctgcagtaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctatttcactttttgcattctacaaactgcataactattatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagacgtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagccttcatcacttgcctcccatagatgaatccgaacctcattacacattagaactgcgaatccatcttcatggtgaaccaaagtgaaacctagtttatcgcaataaaaacctatactctttttaatatccccgactggcaatgccgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacggaattccacctgctgacggagtgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatatatactgattaactaataaggaggacaaaaatgatcttaaatcttgaaaaaaactcattttatacatatcatgtggaaagaggagaggataagatgagccattatgaccagcaatacaatgcaatcattcaaaaaattattgaatcagggatctccgatgaagaatatcaggtccggacgaagtgggattcagacggaacgccggctcatacgctgagcatcatgagcgaaaaaatgcggtttgacaattcggaagtgccgattttgacgacgaagaaggtggcgtggaaaacggccatcaaagagctgctttggatctggcagctgaaatccaatgatgttcaagttttgaacgatatgggggttcatatttgggatcaatggcggcttgaagacggcacaatcggagcagcctacggctatcaattagcgaagaaaaatagaacggttaacgggcagaaagccgatcaagtggattatttgctccatcagttaaagcataatccgtcttcacgcaggcatttgaccatgctgtggaatcccgatgacttggacggcatggccttgacgccctgcgtctatgagacgcaatggtatgtgaaagaaggaaagctctctttggaagtaagagcgagaagcaatgacatggcgcttggcaacccgttcaatgtgttccaatataacgtcttgcagcgcatgatcgcccaagtcctcggttatgagctgggcgaatacatcttcaatatcggcgactgtcacatctatacccgtcacatcgataatttaaacattcagatgaaacgcgagcaatatgaagcgccgaagctgtggatcaacccggacatcaaaaacttttatgactttacaatcgatgattttaaattaattgactataagcacggcgacaagctgacatttgaagttgccgtataagcttccaggcatcaaataaaacgaaaggctcagtcgaaagactgggcctttcgttttatctgttgtttgtcggtgaacgctctctactagagtcacactggctcaccttcgggtgggcctttctgcgtttatacgctcagatgacgcaggtgctgcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggac

SEQ ID NO: 46 - DNA, 24 bp, synthetic construct
  misc_feature 1..24: note = Synthetic
  source 1..24: mol_type = other DNA
gattaactaataaggaggacaaac

SEQ ID NO: 47 - DNA, 21 bp, synthetic construct
  misc_feature 1..21: note = Synthetic
  source 1..21: mol_type = other DNA
gctcttaaggaggattttaga

SEQ ID NO: 48 - DNA, 21 bp, synthetic construct
  misc_feature 1..21: note = Synthetic
  source 1..21: mol_type = other DNA
ggtggaaaggaggtgatcgac

SEQ ID NO: 49 - DNA, 22 bp, synthetic construct
  misc_feature 1..22: note = Synthetic
  source 1..22: mol_type = other DNA
ggtgggaaggaggacattcgac

SEQ ID NO: 50 - DNA, 24 bp, synthetic construct
  misc_feature 1..24: note = Synthetic
  source 1..24: mol_type = other DNA
tgacatgaaaggaagtatttgaaa

SEQ ID NO: 51 - DNA, 20 bp, synthetic construct
  misc_feature 1..20: note = Synthetic
  source 1..20: mol_type = other DNA
ggtgggaaggaggtgatcca

SEQ ID NO: 52 - DNA, 22 bp, synthetic construct
  misc_feature 1..22: note = Synthetic
  source 1..22: mol_type = other DNA
ggtgggaaggagggggttcgac

SEQ ID NO: 53 - DNA, 20 bp, synthetic construct
  misc_feature 1..20: note = Synthetic
  source 1..20: mol_type = other DNA
ggtgggaaggaggaactact